(19) United States
(12) Patent Application Publication
(10) Pub. No.: US 2013/0165339 A1
(43) Pub. Date: Jun. 27, 2013

(54) SAPP-ALPHA AS A BIOMARKER FOR PREDICTION OF INFLAMMATORY AND AUTOIMMUNE-RELATED DISORDERS

(71) Applicants: Jun Tan, Tampa, FL (US); Demian Obregon, Tampa, FL (US); Antoinette Bailey, Tampa, FL (US)

(72) Inventors: Jun Tan, Tampa, FL (US); Demian Obregon, Tampa, FL (US); Antoinette Bailey, Tampa, FL (US)

(73) Assignee: UNIVERSITY OF SOUTH FLORIDA, TAMPA, FL (US)

(21) Appl. No.: 13/704,921

(22) PCT Filed: Jun. 30, 2011
(86) PCT No.: PCT/US11/42648

§ 371 Date: Mar. 1, 2013

Related U.S. Application Data
(60) Provisional application No. 61/360,076, filed on Jun. 30, 2010.

(51) Int. Cl. G01N 33/68

(52) U.S. Cl. CPC G01N 33/68
USPC 506/9; 436/501; 435/7.92; 435/7.9

(57) ABSTRACT

The subject invention pertains to the use of amyloid precursor protein-alpha (sAPP-α) as a biomarker for prediction of a subject's risk of developing inflammatory and/or autoimmune-related disorders. In addition, the present invention provides methods for optimizing vaccine schedules and compositions, thereby preventing or reducing the risks of vaccine-induced inflammatory and/or autoimmune-related disorders.

### CROSS-REFERENCE TO RELATED APPLICATION

This application claims the benefit of U.S. provisional application Ser. No. 61/360,076, filed Jun. 30, 2010, which is herein incorporated by reference in its entirety.

### BACKGROUND OF THE INVENTION

Autoimmune diseases affect over 40 million individuals in the United States. Unfortunately, a significant proportion of autoimmune diseases are triggered by iatrogenic factors. Among these factors, childhood vaccinations may play a causal role in the development and regression of a variety of autoimmune diseases in genetically predisposed individuals. For instance, it is suggested that the pathogenesis of a variety of autoimmune diseases, including autism, Guillain-Barré syndrome, multiple sclerosis, rheumatoid arthritis, systemic lupus erythematosus, postvaccinal encephalomyelitis, seizure, paralysis, mental retardation, attention-deficit hyperactivity disorder (ADHD), and dyslexia, might be linked to autoimmune over-reactions triggered by vaccinations.

Although vaccinations are considered as the most effective approach for preventing diseases and infections, the current “one-size-fits-all” vaccination practice poses a lurking threat of developing chronic autoimmune diseases in later life. There has not been any effective method for identification of individuals who are at risk of inflammatory and/or autoimmune diseases. Additionally, there has not been any effective method for providing customized vaccine compositions and schedules that can prevent or reduce such risks. Therefore, a need exists in the art for methods to predict risk of autoimmune disorders and to provide optimized vaccine schedules and compositions that can prevent or reduce vaccine-induced autoimmune disorders.

### BRIEF SUMMARY OF THE INVENTION

The aforementioned need is satisfied by the present invention, utilizing soluble amyloid precursor protein-alpha (sAPP-α) as a biomarker for prediction of a subject's risk of developing inflammatory and/or autoimmune-related disorders. In an embodiment, the present method predicts a subject's risk of developing inflammatory and/or autoimmune-related disorders associated with vaccinations.

In another aspect, the present invention provides methods for optimizing vaccine schedules and compositions, thereby preventing or reducing the risks of vaccine-induced inflammatory and/or autoimmune-related disorders.

Preferably, sAPP-α level of an infant is determined using methods of the present invention prior to its first administration of a vaccine composition. This allows for early detection of infants who are at risk of developing inflammatory and/or autoimmune disorders triggered by vaccinations. Accordingly, alternative vaccine schedules and/or compositions might be provided to prevent or reduce such risk.

In an embodiment, the method of the present invention further involves determination of the level of a second biomarker that is associated with immune function and/or inflammation. Exemplified second biomarkers include, but are not limited to, IL-1, IL-2, IL-3, IL-4, IL-6, IL-7, IL-18, IL-8, CD4, CD8, TNF-α, TNF-β; LAF, BSF-1, IFN-α, IFN-β, IFN-γ; TGF β; nitric oxide, nitric oxide synthase, IgA, IgG, IgM, IgD, and IgE.

Methods of the present invention are useful for predicting, preventing, or reducing risks of inflammatory and/or autoimmune-related disorders, including but not limited to, autism, multiple sclerosis (MS), autoimmune thyroid disease, psoriasis, Guillain-Barré syndrome, systemic lupus erythematosus, postvaccinal encephalomyelitis, seizure, paralysis, mental retardation, attention-deficit hyperactivity disorder (ADHD), and dyslexia.

### BRIEF DESCRIPTION OF THE DRAWINGS

FIG. 1 shows the plasmid and human sAPP-alpha gene used to generate the transgenic sAPP-alpha overexpressing mice. Each mouse has about 2-6 copies of the human APP 695 isoform-derived sAPP-alpha gene that is under the control of the prion promoter.

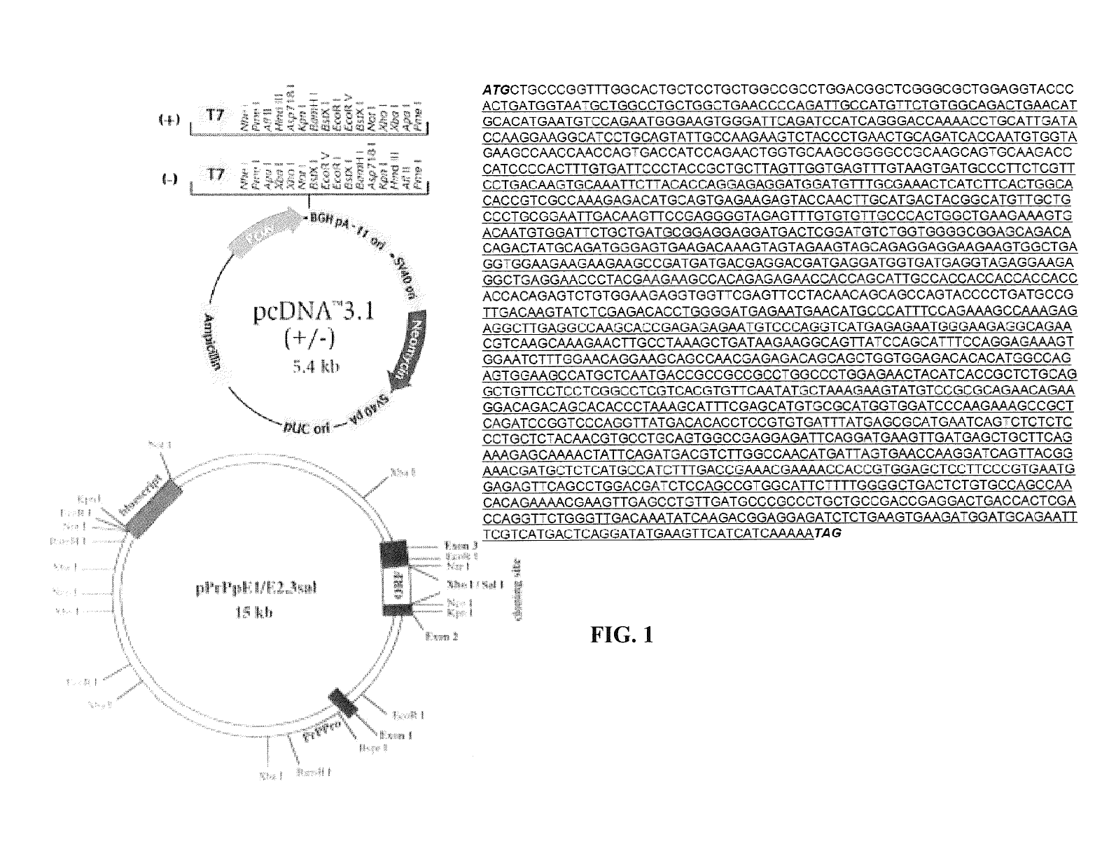

FIG. 2 characterizes sAPP-alpha constructs.

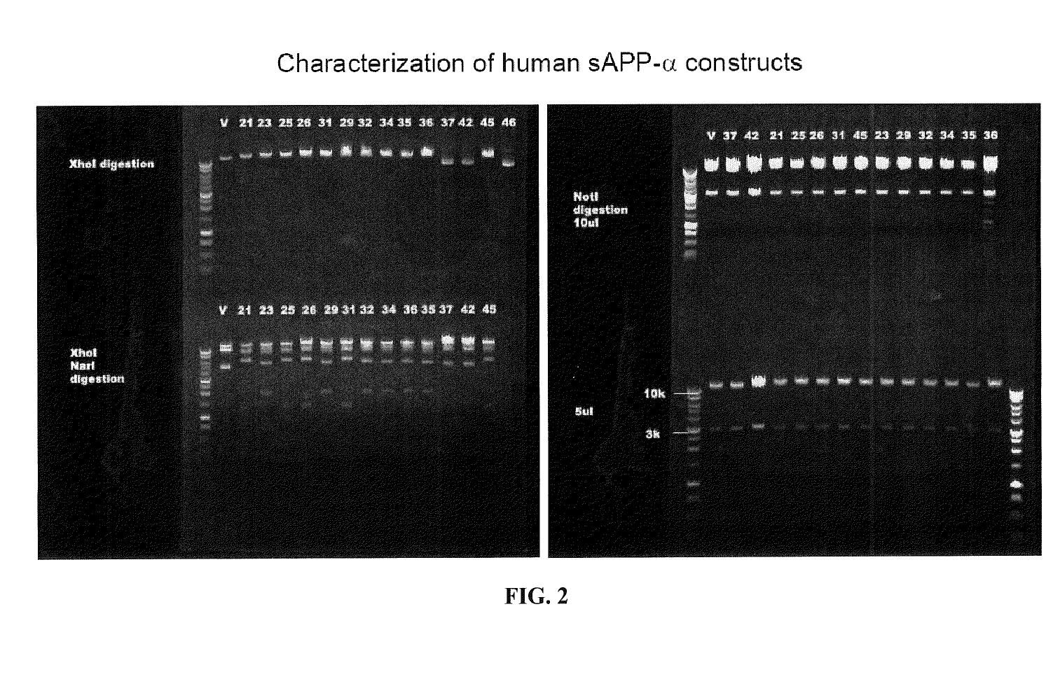

FIG. 3 shows the expression of human sAPP-alpha gene in the transgenic mice. (A) RT-PCR results; (B) Western analysis using anti-human sAPP-alpha antibody 6E10. The results show the presence of human sAPP-alpha protein in transgenic mouse brain tissue.

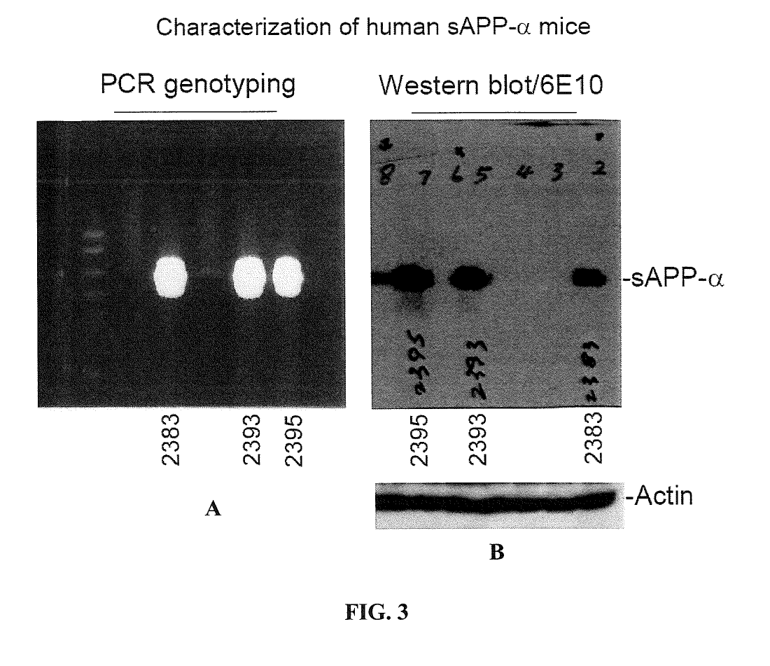

FIG. 4 shows the presence of sAPP-alpha protein in transgenic mouse brain tissue. The results are obtained by Western analysis using anti-human sAPP-alpha antibody 6E10.

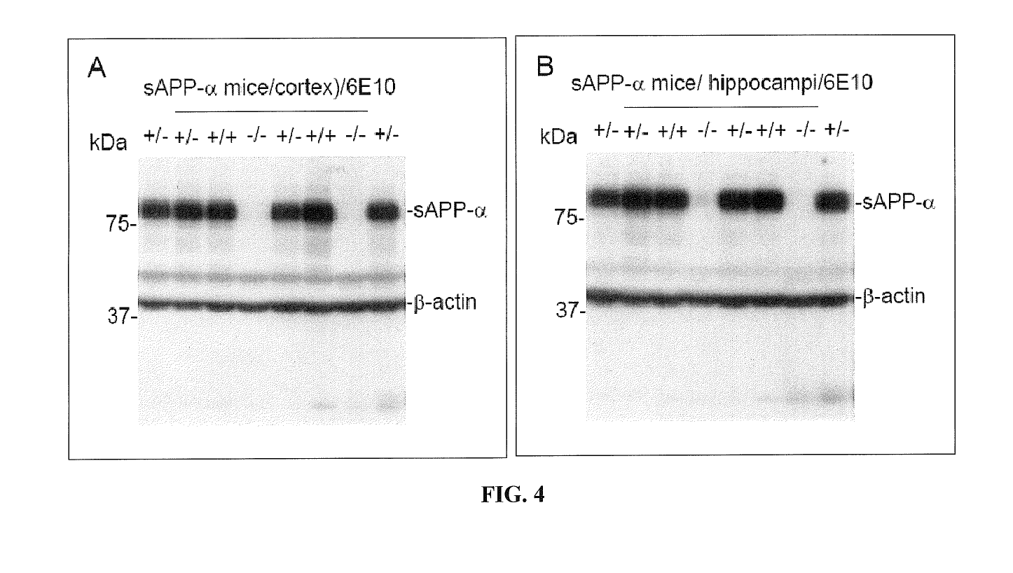

FIG. 5A shows flow cytometry analysis for the average number of thymocytes and splenocytes in sAPP-alpha overexpressing mice. FIG. 5B shows IFN-γ production by splenocytes after ConA treatment.

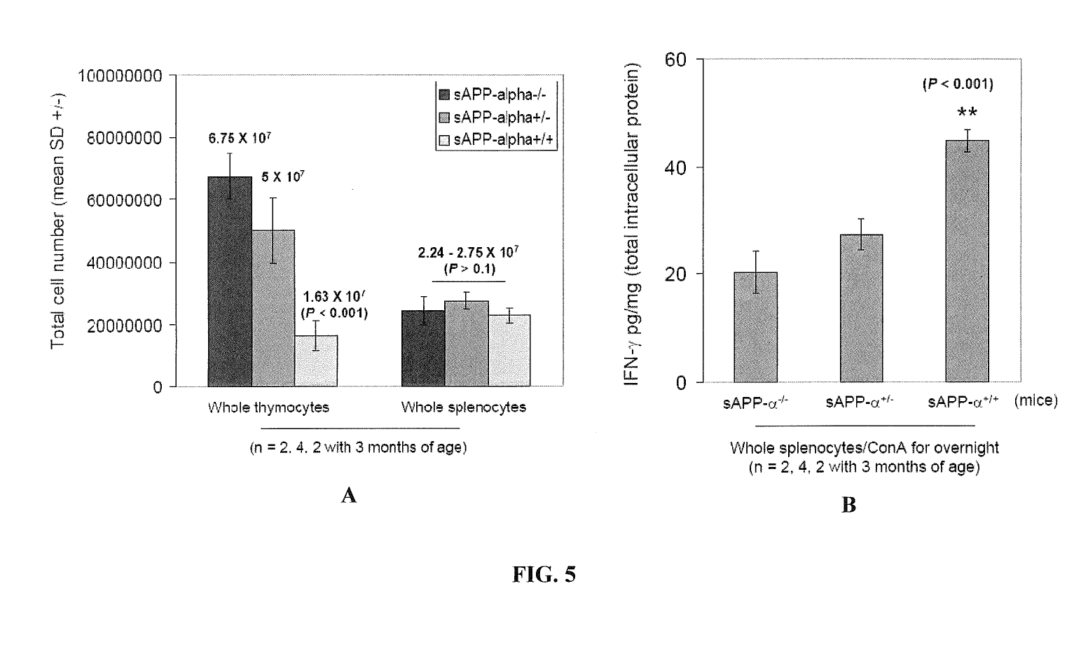

FIG. 6 shows flow cytometry analysis for CD3+ splenocytes isolated from sAPP-alpha overexpressing mice.

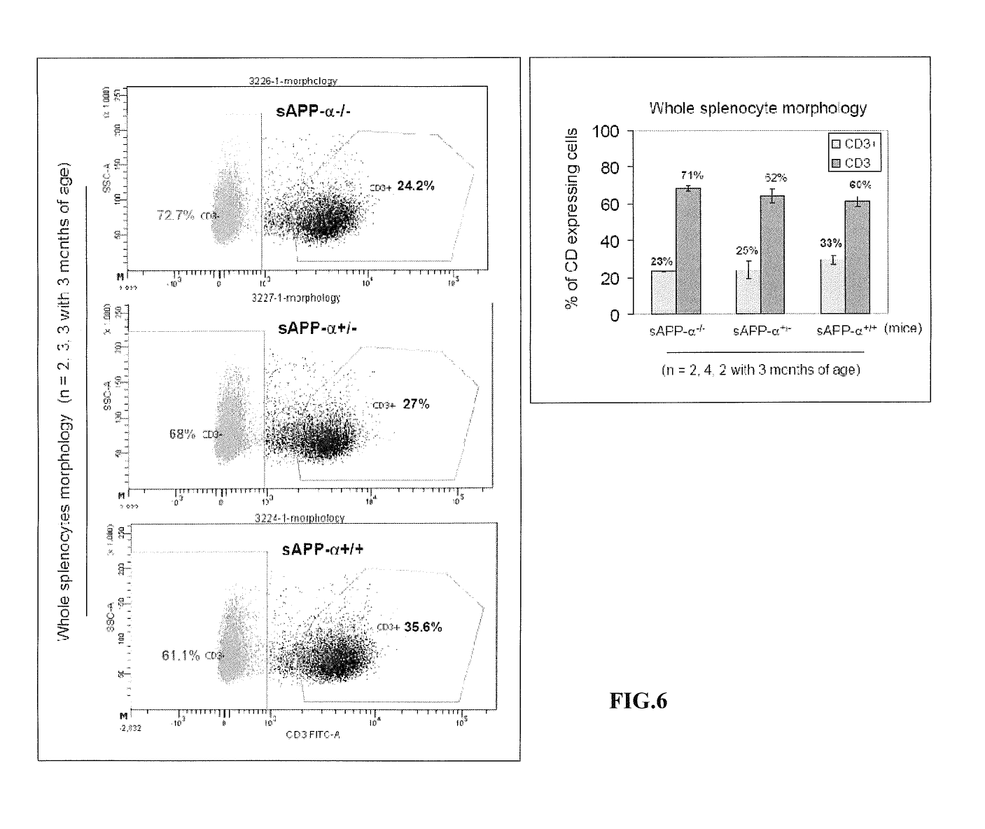

FIG. 7 shows flow cytometry analysis for CD3+/CD19+ (B-cells); CD3+/CD8+/CD4+ (T-cells) splenocytes isolated from sAPP-alpha overexpressing mice. (A) whole splenocyte morphology; (B) CD3+/splenocytes; (C) whole splenocytes morphology.

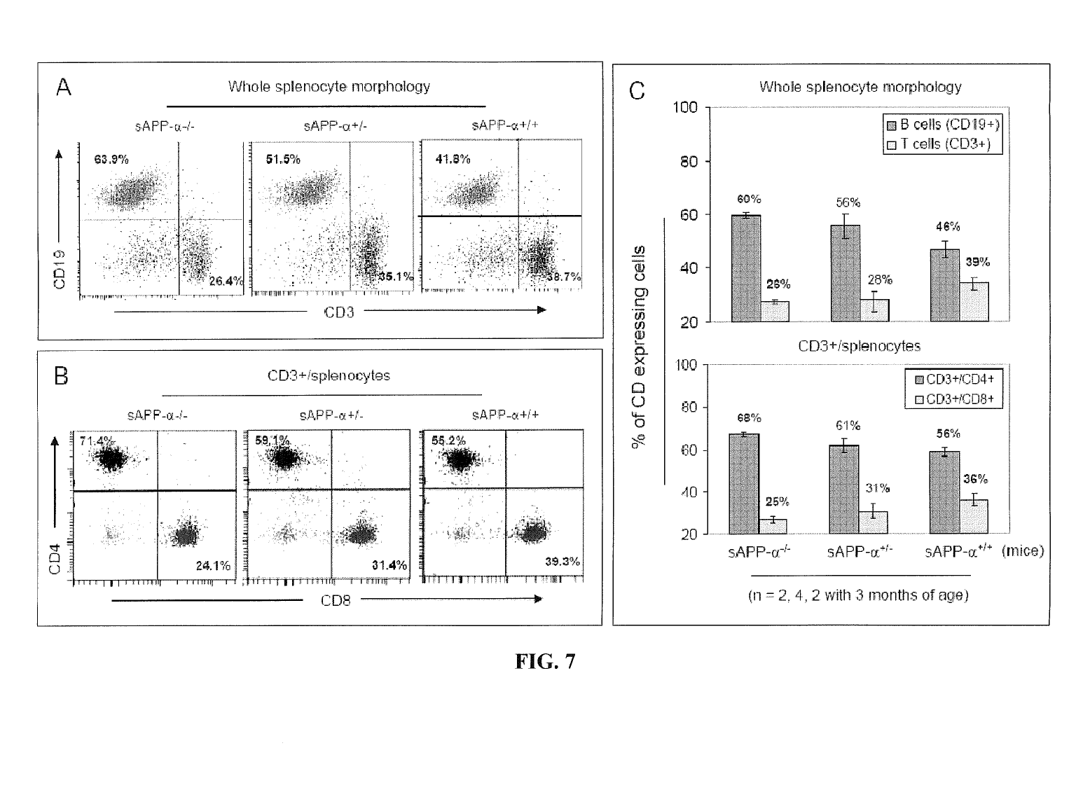

FIG. 8 shows flow cytometry analysis for activated effector memory T-cells (CD3/CD44/CD25) and non-activated effector memory T-cells (CD3/CD44/CD4 or 8) from splenocytes isolated from sAPP-alpha overexpressing mice.

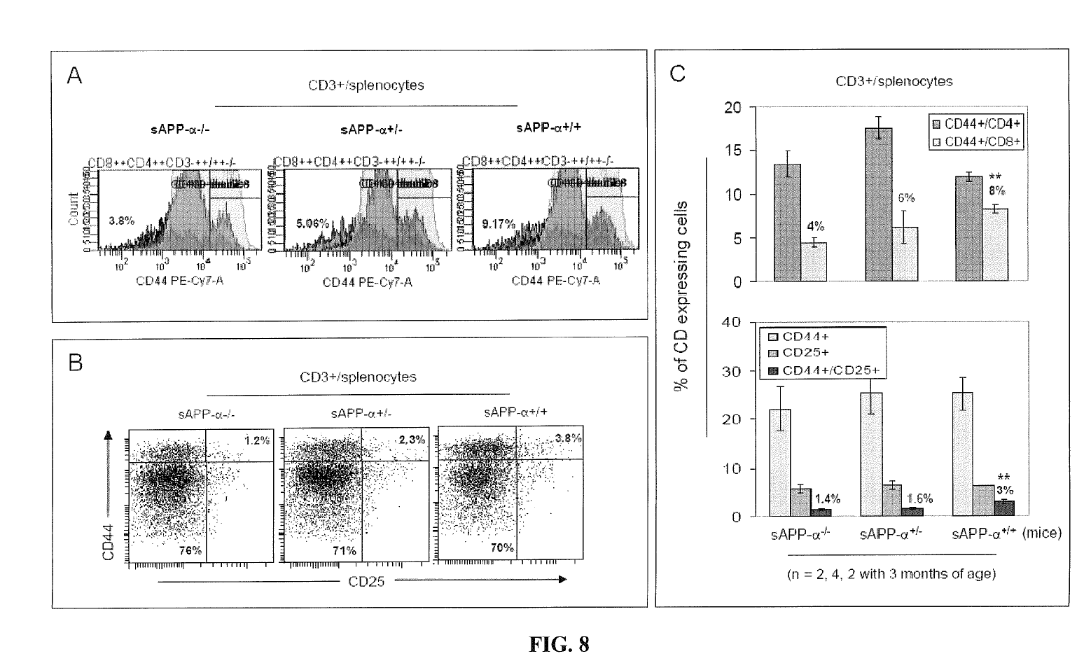

FIG. 9 shows flow cytometry analysis for lymphocytes (CD3+) in thymus tissues isolated from sAPP-alpha overexpressing mice.

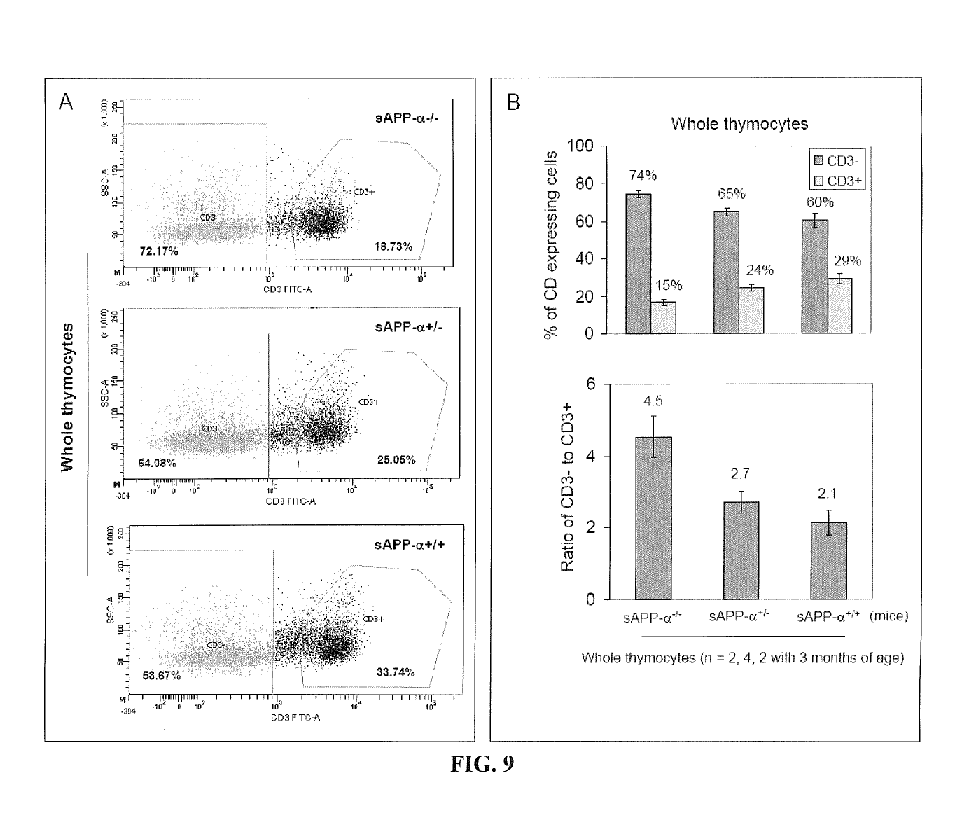

FIG. 10 illustrates that T-cell development is a key component of the immune system.

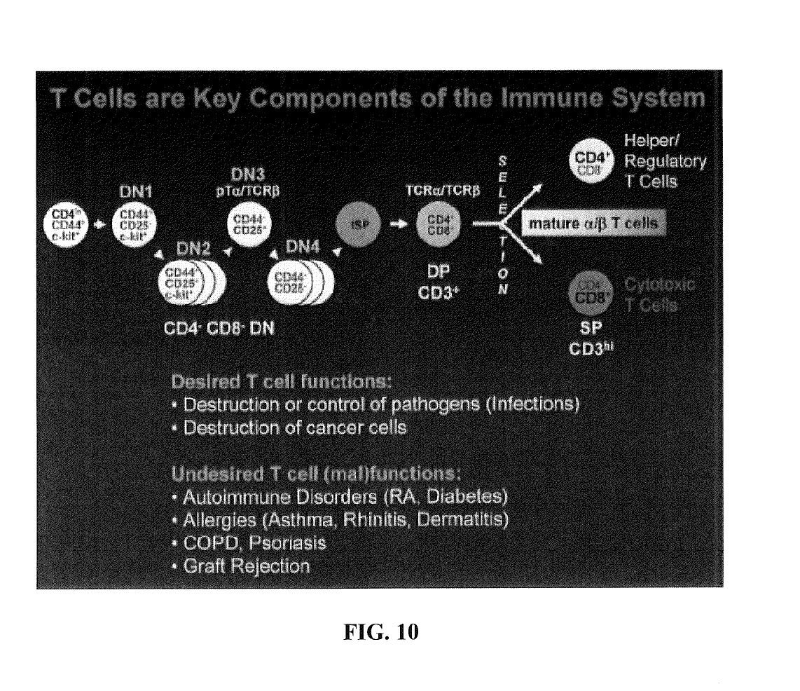

FIG. 11 shows flow cytometry analysis for T-cells (CD4 and/or 8) and for activated effector memory T-cells (CD44/CD25) from thymocytes isolated from sAPP-alpha overexpressing mice.

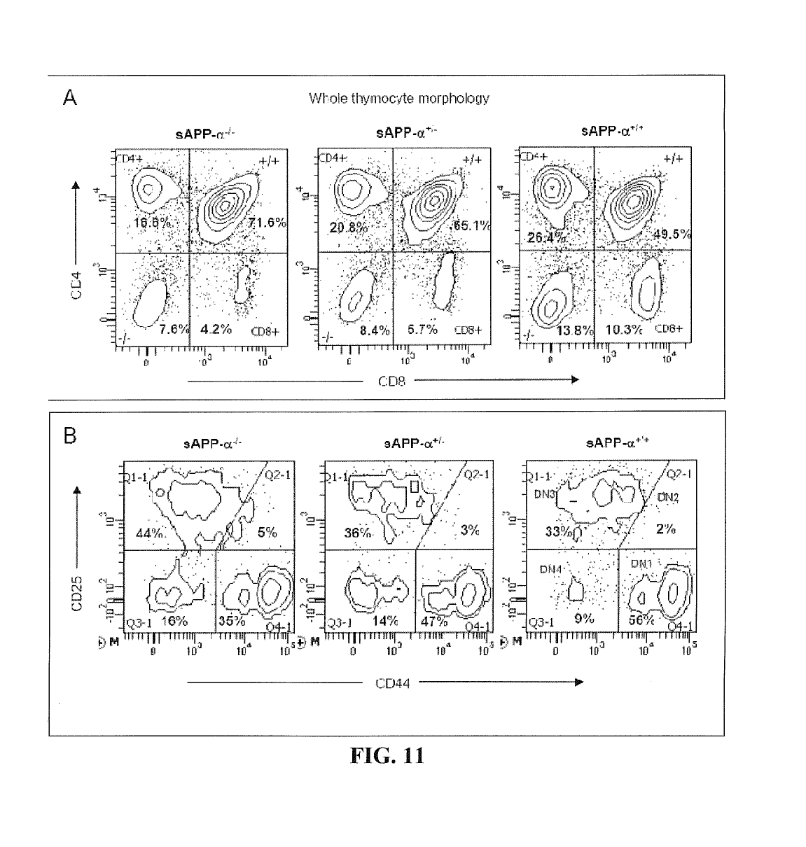

FIG. 12 shows flow cytometry analysis for T-cell markers from thymocytes isolated from sAPP-alpha overexpressing mice.

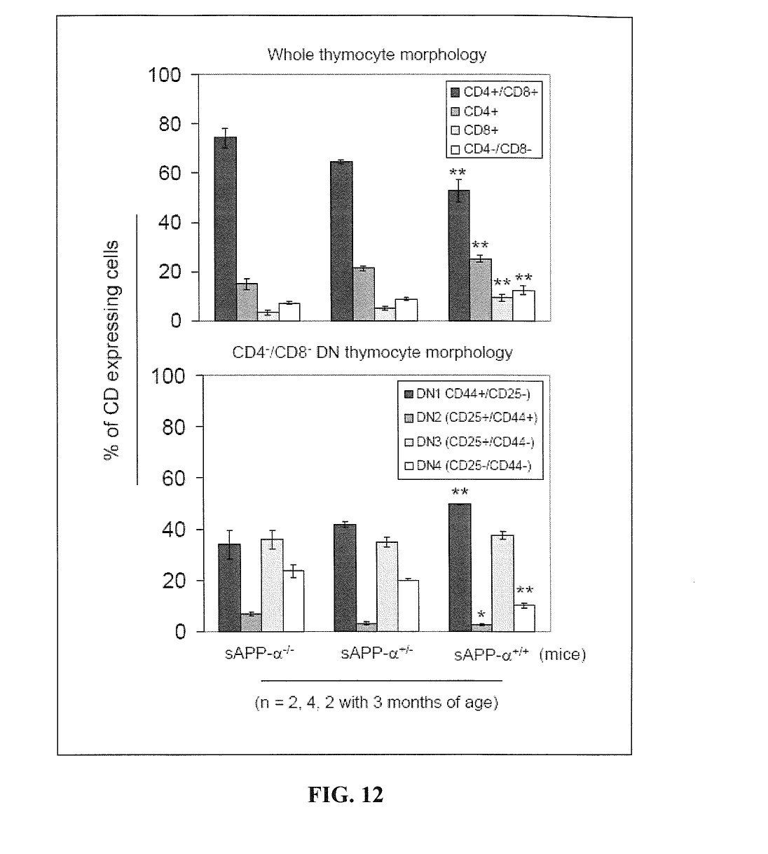

FIG. 13 shows Western analysis of sAPP-alpha levels in splenocytes isolated from sAPP-alpha overexpressing mice. (A) The splenocytes are not treated with ConA, and the Western analysis is performed using anti-human sAPP-alpha antibody 6E10. (B) The splenocytes are treated for 24 hours with ConA, and the Western analysis is performed using anti-human sAPP-alpha antibody 6E10.

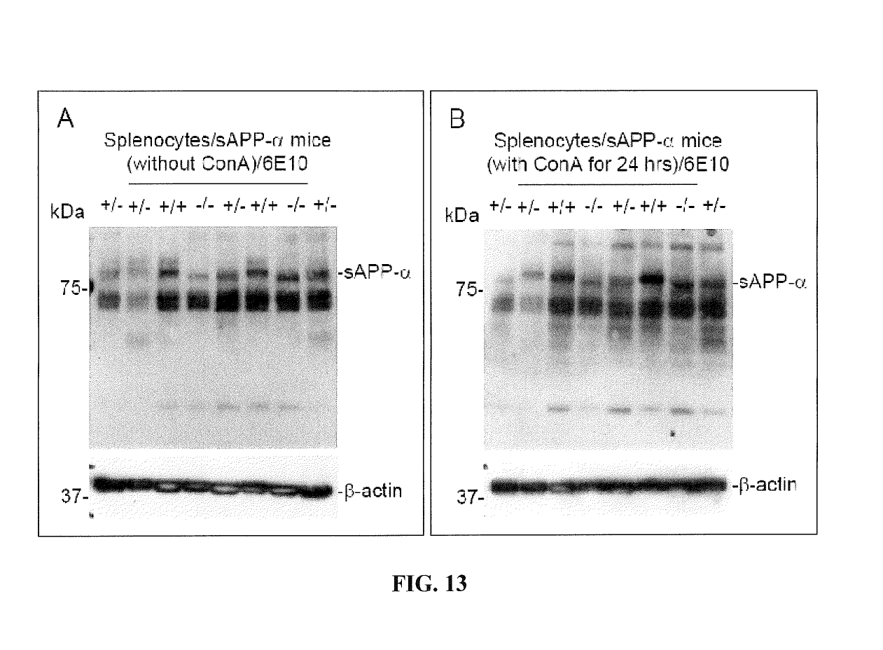

FIG. 14 shows Western analysis of sAPP-alpha level in thymocytes of sAPP-alpha overexpressing mice. The Western analysis is performed using anti-human sAPP-alpha antibody 6E10.

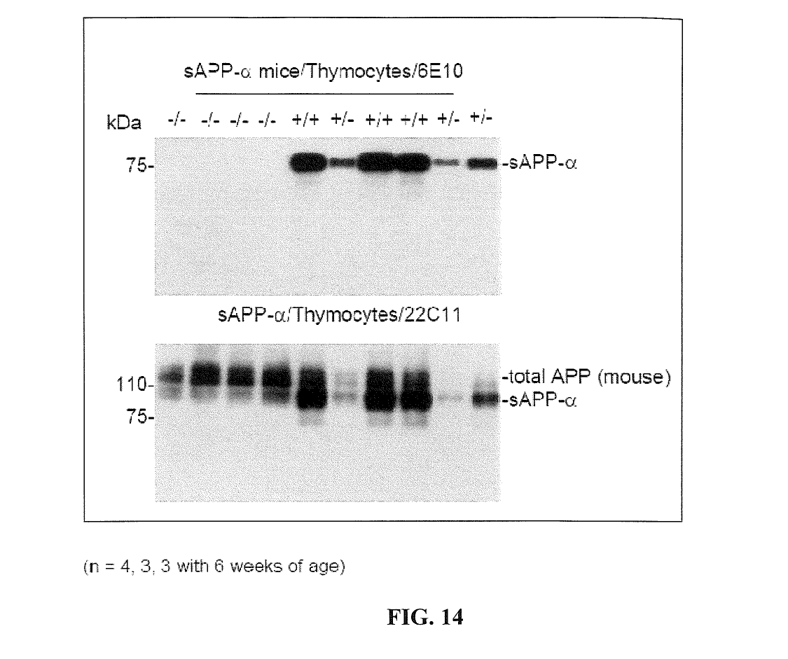

FIG. 15 shows immunochemistry staining (IHC) analysis of sAPP-alpha in thymus tissues isolated from sAPP-alpha overexpressing mice, using anti-human sAPP-alpha antibody 6E10.

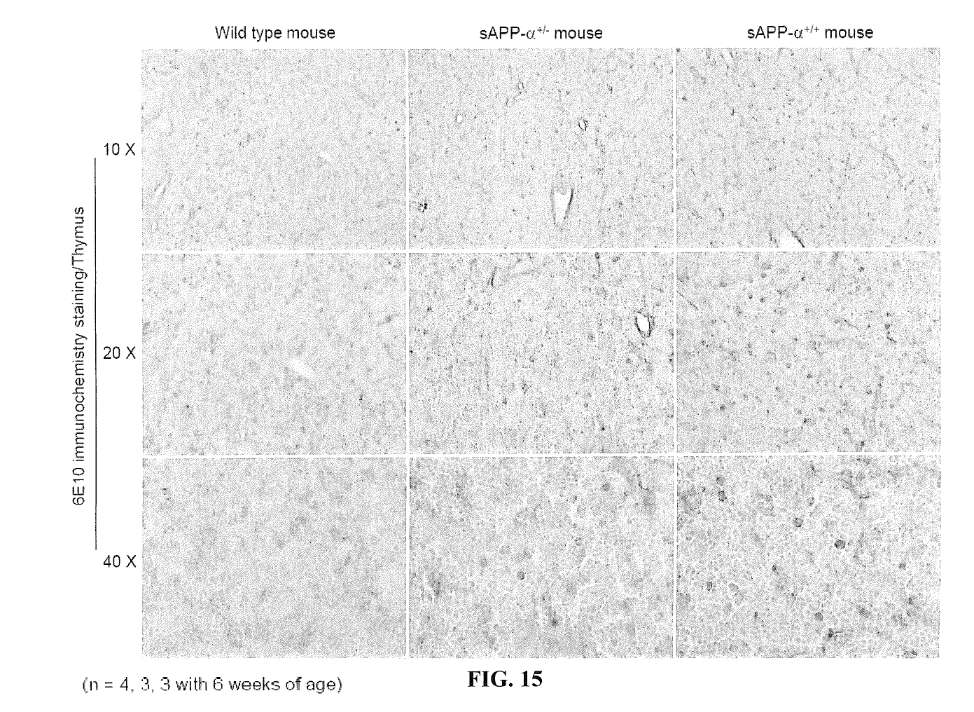

FIG. 16 shows Western analysis of the level of sAPP-alpha, total caspase 3, and cleaved caspase 3 in thymus tissues isolated from sAPP-alpha overexpressing mice, using anti-human sAPP-alpha antibody 6E10 and antibodies against total and activated (cleaved) caspase 3.

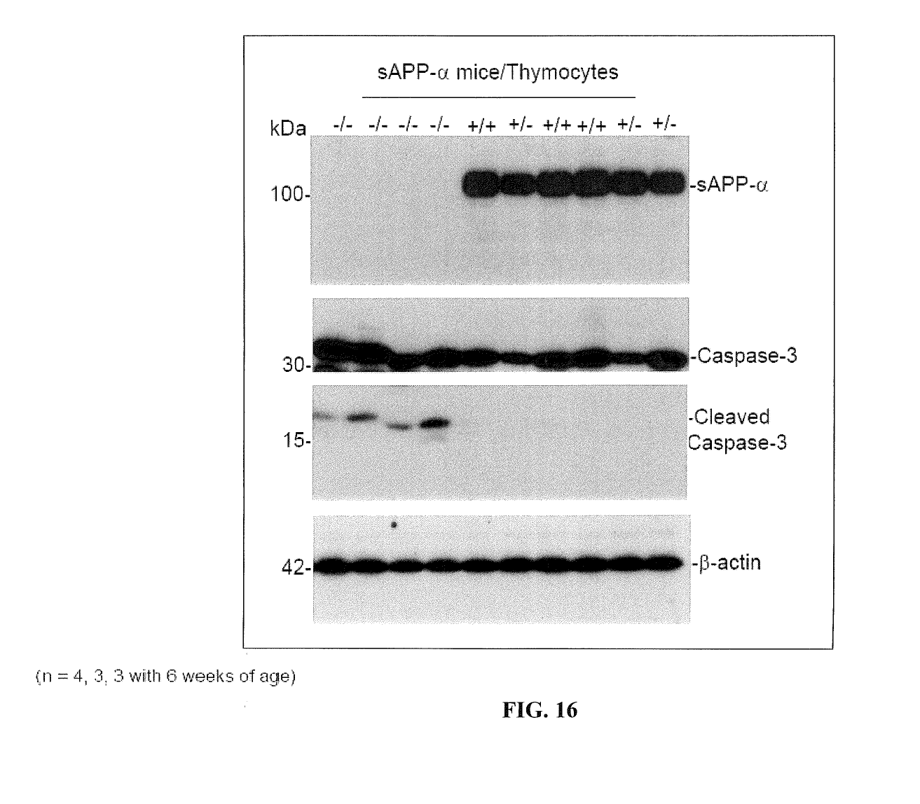

FIG. 17 shows immunochemistry staining (IHC) analysis of the activated (cleaved) caspase 3 in thymus tissues isolated from sAPP-alpha overexpressing mice, using antibodies against activated (cleaved) caspase 3 of thymus tissues isolated from sAPP-alpha overexpressing mice.

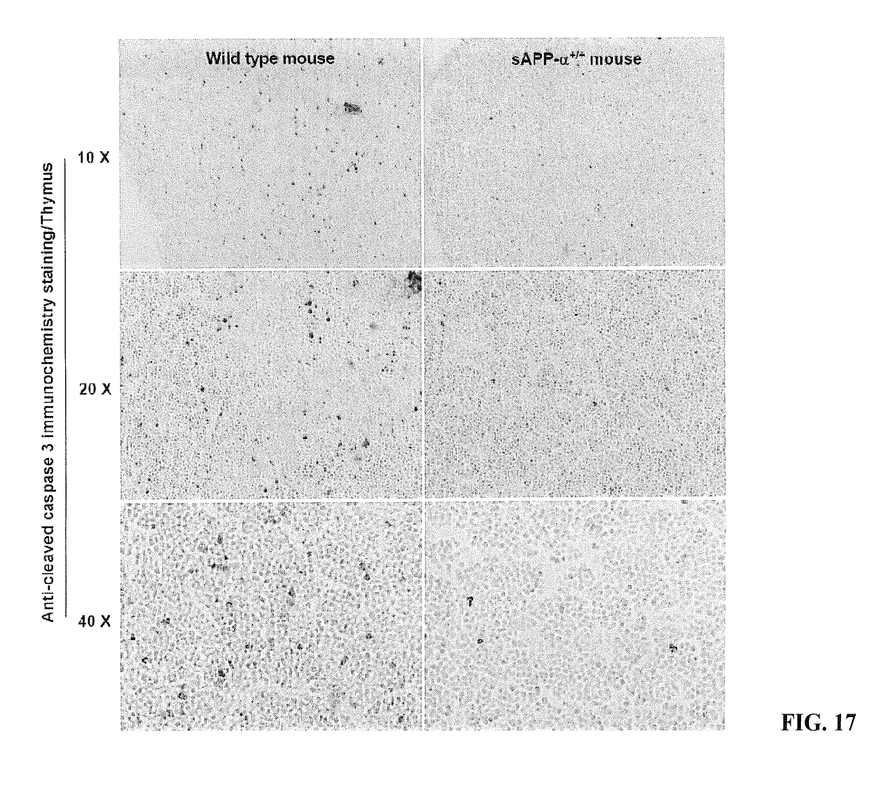

FIG. 18 shows immunochemistry staining (IHC) analysis of the activated (cleaved) caspase 3 in thymus tissues isolated from sAPP-alpha overexpressing mice, using antibodies against activated (cleaved) caspase 3.

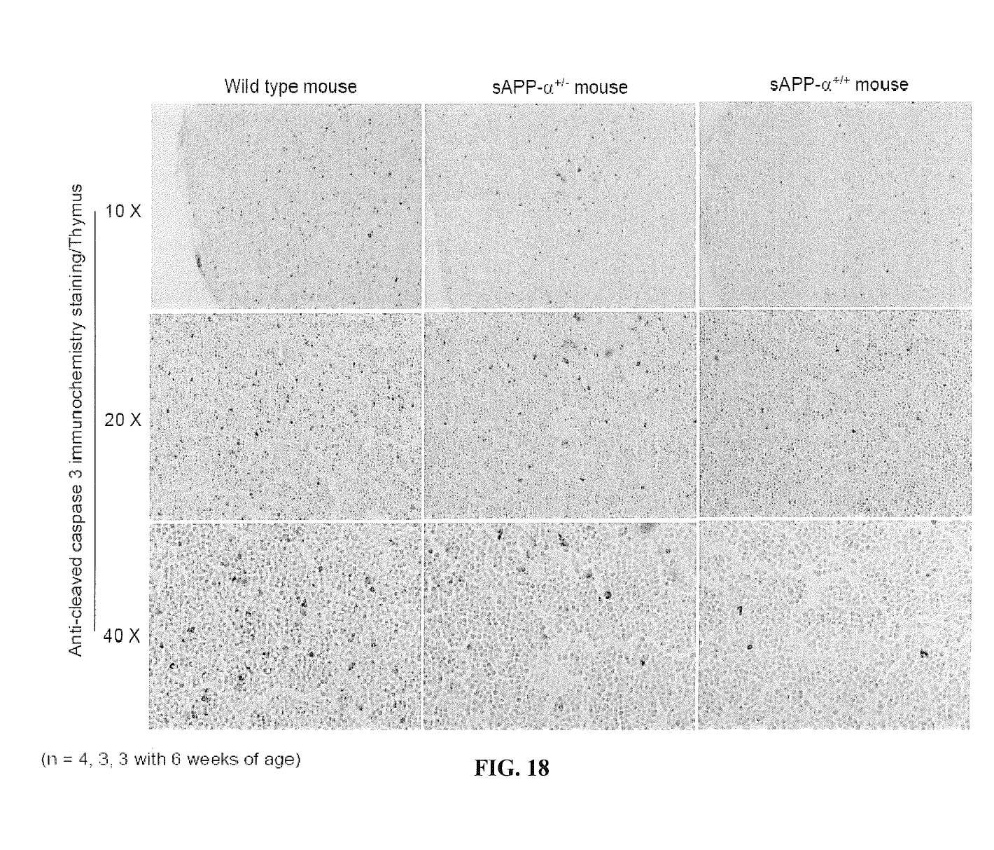

FIG. 19 shows TUNEL analysis for apoptosis in thymic tissues isolated from sAPP-alpha overexpressing mice.

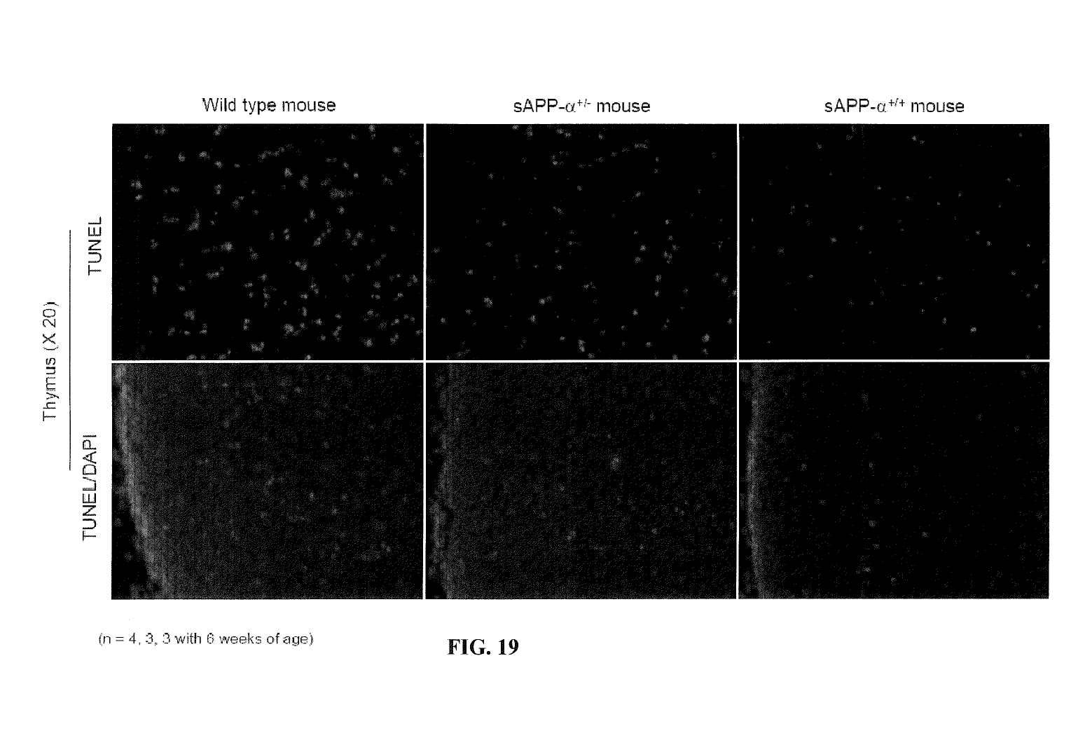

FIG. 20 shows enzyme-linked immunosorbent assay (ELISA) analysis for levels of plasma inflammatory cytokines IL-6 and TNF-alpha in sAPP-alpha overexpressing mice.

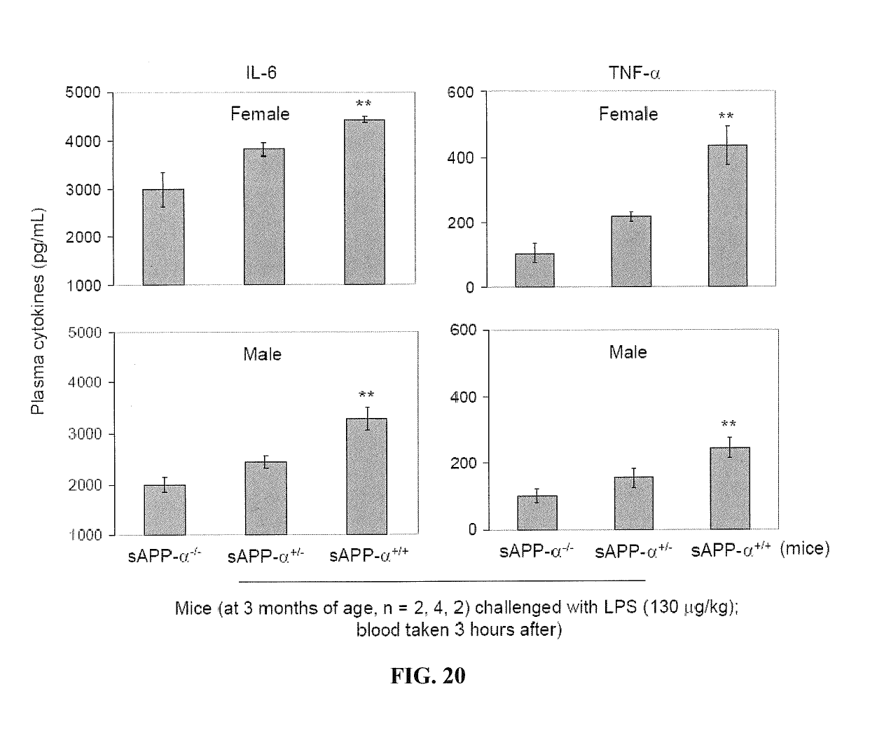

FIG. 21 shows Western analysis of microtubule-associated protein 2 (MAP2) (an early neuron marker) and neuronal nuclei (NeuN) (a mature neuron maker) levels in CNS tissues isolated from sAPP-alpha overexpressing mice. The Western analysis is performed using anti-human MAP2 and NeuN antibodies.

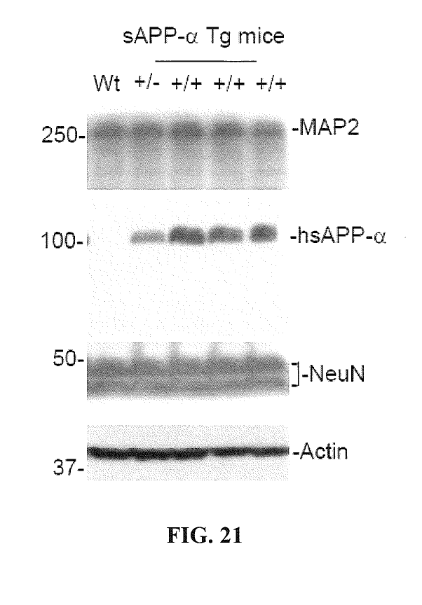

FIG. 22 shows immunochemistry staining (IHC) for NeuN in CNS tissues isolated from sAPP-alpha overexpressing mice. The results show that, after LPS challenge, the sAPP-alpha overexpressing mice have neuron loss when compared to the control littermates.

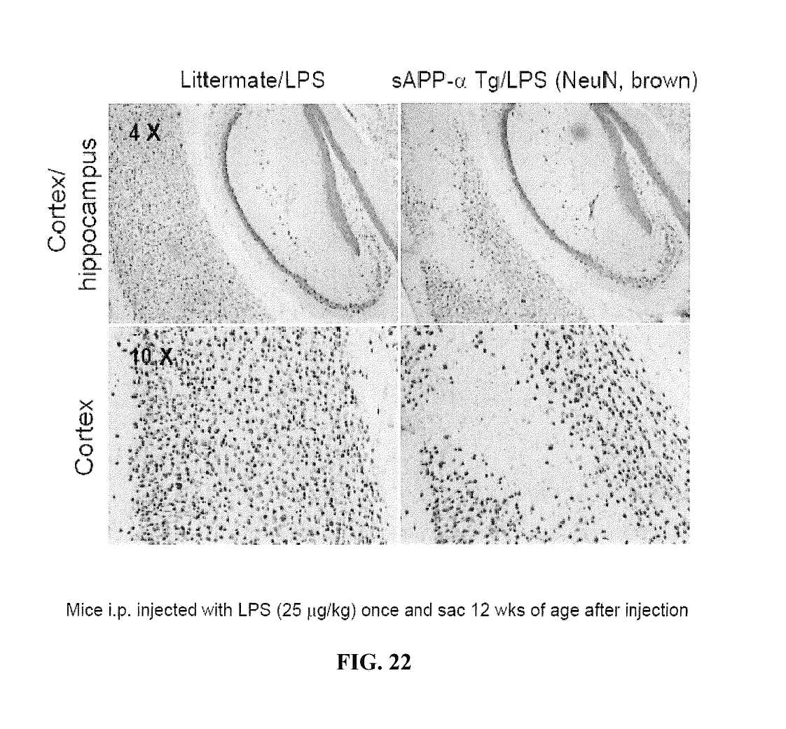

FIG. 23 shows Nissl stain of cortex tissues isolated from sAPP-alpha overexpressing mice. The results show that, after LPS challenge, the sAPP-alpha overexpressing mice have abnormal neurons and increased glial cells when compared to the control littermates.

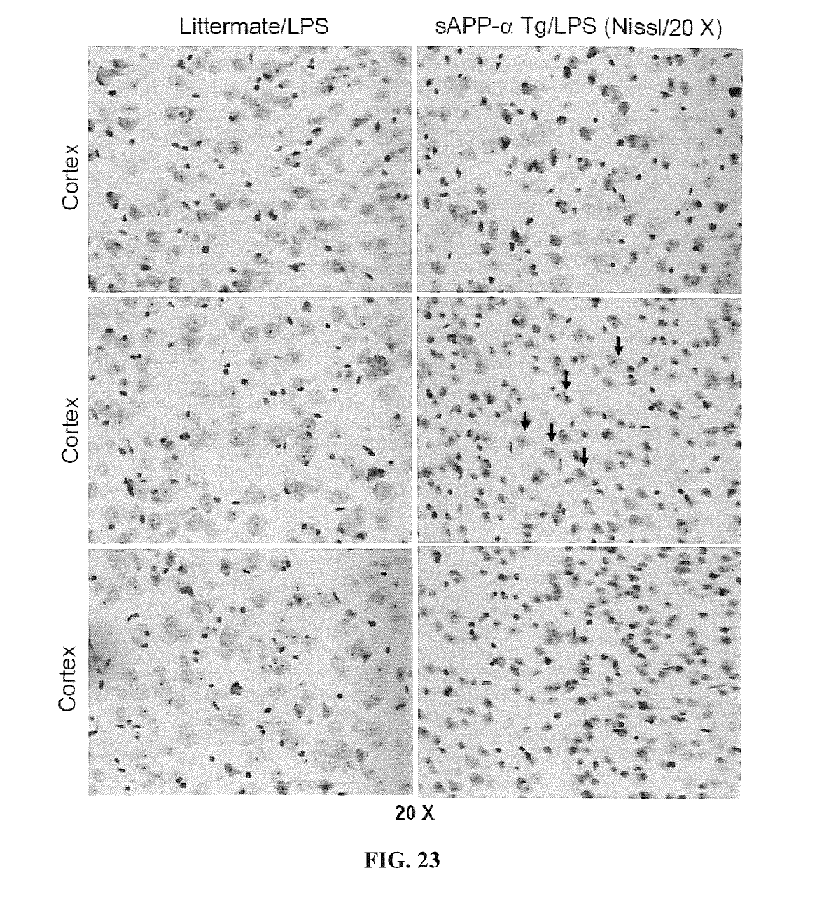

FIG. 24 shows Nissl stain of cortex tissues isolated from sAPP-alpha overexpressing mice. The results show that, after LPS challenge, the sAPP-alpha overexpressing mice have abnormal neurons and increased glial cells when compared to the control littermates.

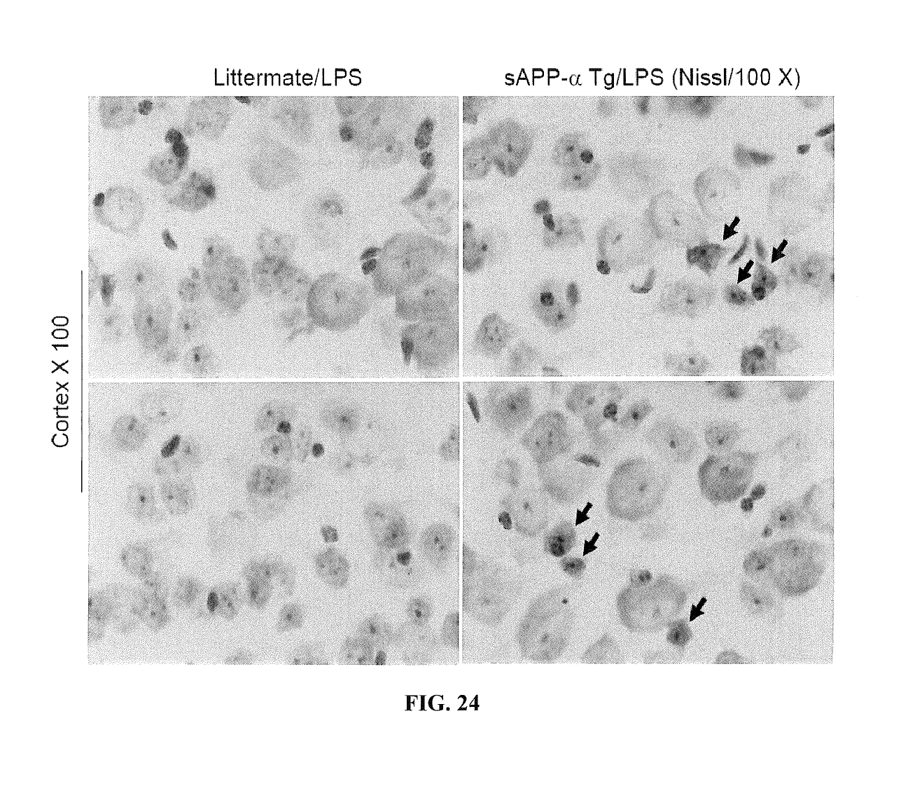

FIG. 25 shows Nissl stain and immunochemistry staining (IHC) analysis for NeuN expression in cortex tissues isolated from sAPP-alpha overexpressing mice. The results show that, after LPS challenge, the sAPP-alpha overexpressing mice have abnormal neurons and increased glial cells when compared to the control littermates.

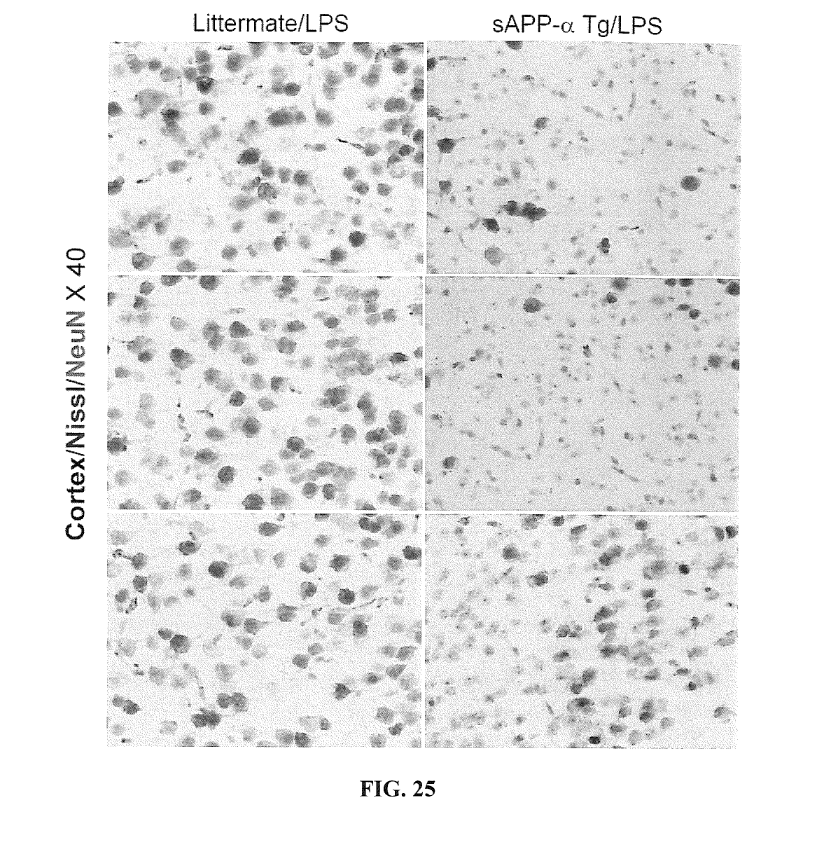

FIG. 26 shows Nissl stain and immunochemistry staining (IHC) analysis for NeuN expression in white matter isolated from sAPP-alpha overexpressing mice. The results show that, after LPS challenge, the sAPP-alpha overexpressing mice have abnormal neurons and increased glial cells when compared to the control littermates.

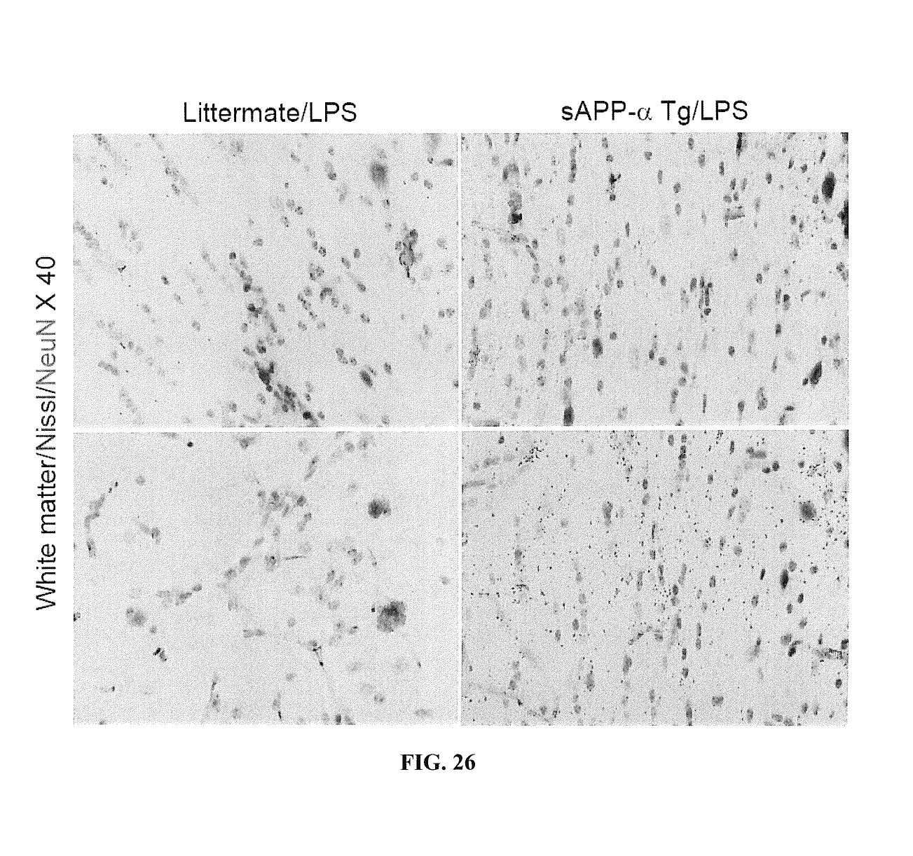

FIG. 27 shows immunochemistry staining (IHC) analysis for MAP2 in areas of neuron loss after sAPP-alpha overexpressing mice receive LPS challenge.

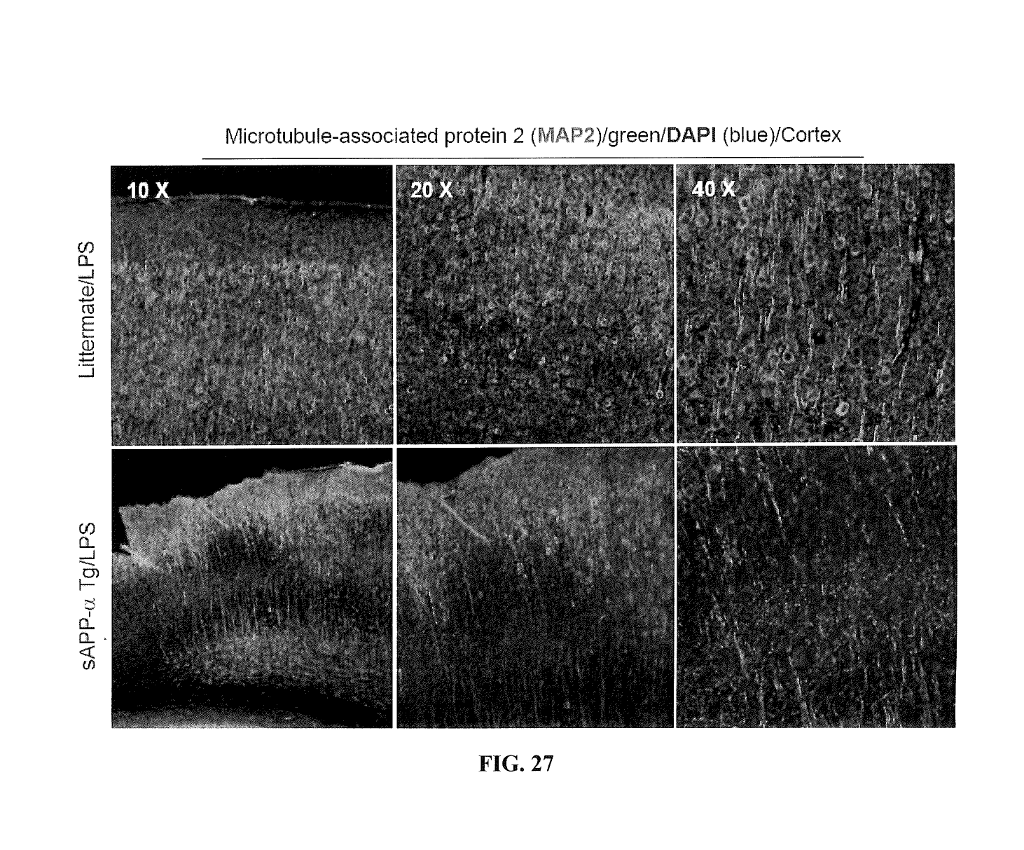

FIG. 28 shows immunochemistry staining (IHC) analysis for MAP2 in areas of neuron loss after sAPP-alpha overexpressing mice receive LPS challenge.

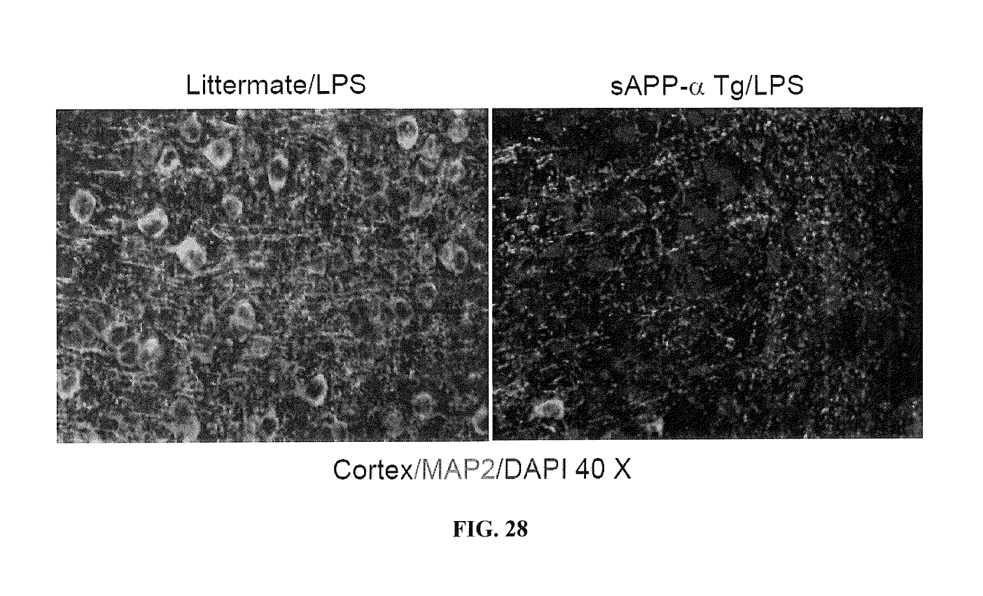

FIG. 29 shows immunochemistry staining (IHC) analysis for NeuN and glial fibrillary acidic protein (GFAP) in areas of neuron loss after sAPP-alpha overexpressing mice receive LPS challenge.

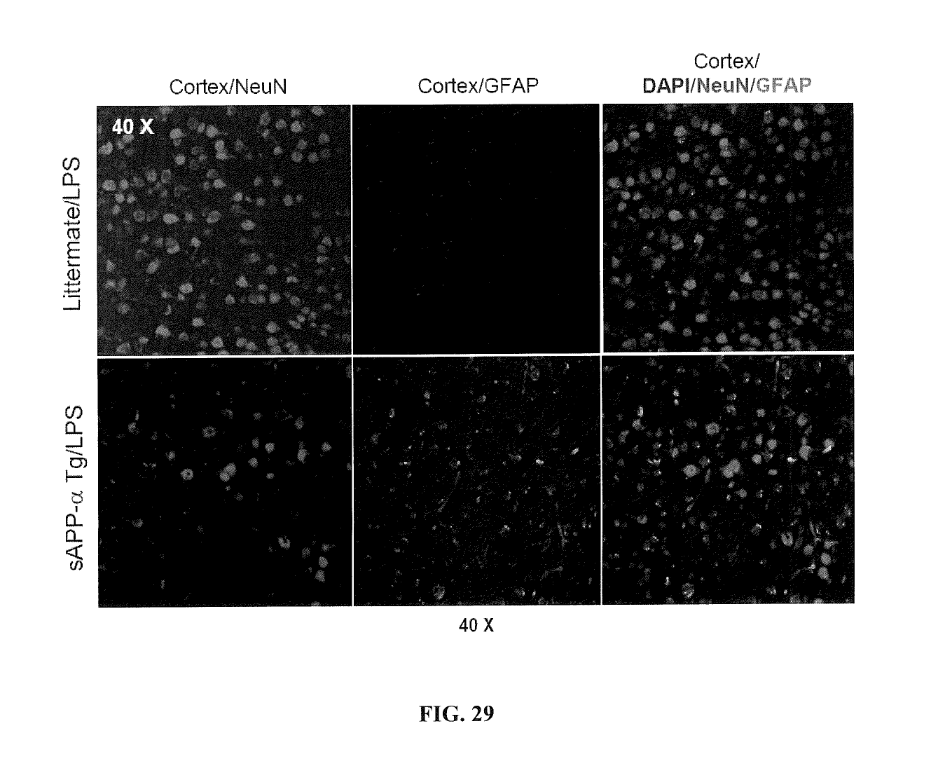

FIG. 30 shows immunochemistry staining (IHC) analysis for NeuN and ionized calcium binding adaptor molecule 1 (Iba1) in areas of neuron loss after sAPP-alpha overexpressing mice receive LPS challenge.

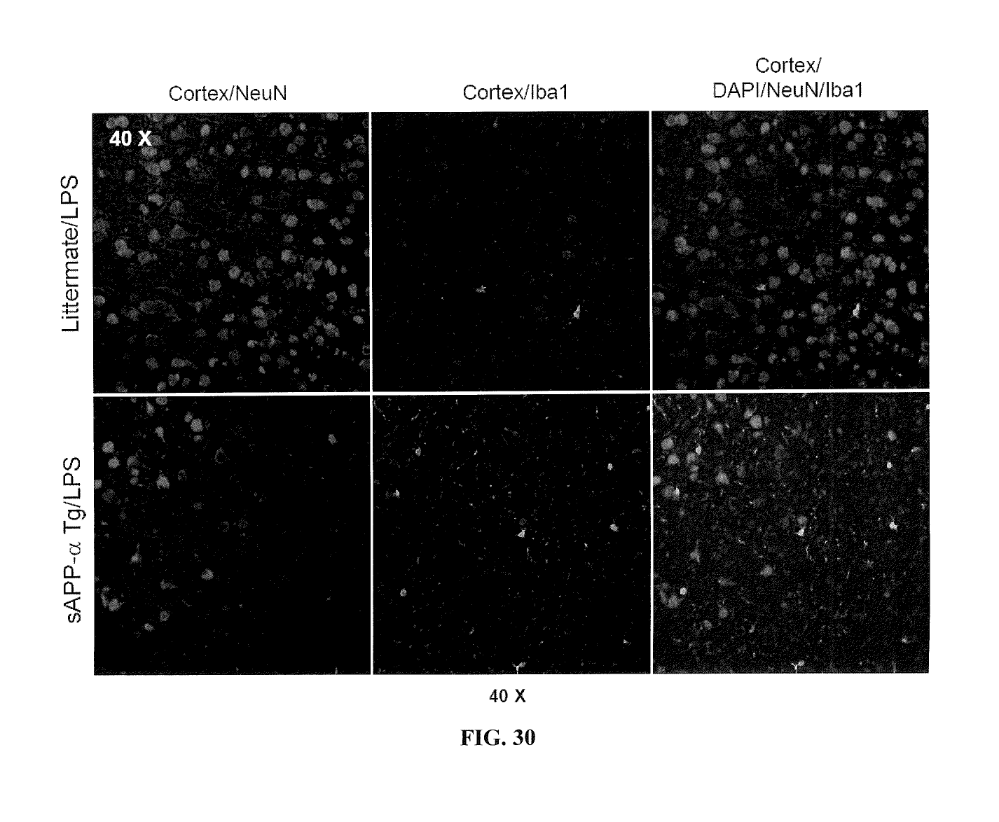

FIG. 31 shows immunochemistry staining (IHC) analysis for beta-tubulin in areas of neuron loss after sAPP-alpha overexpressing mice receive LPS challenge.

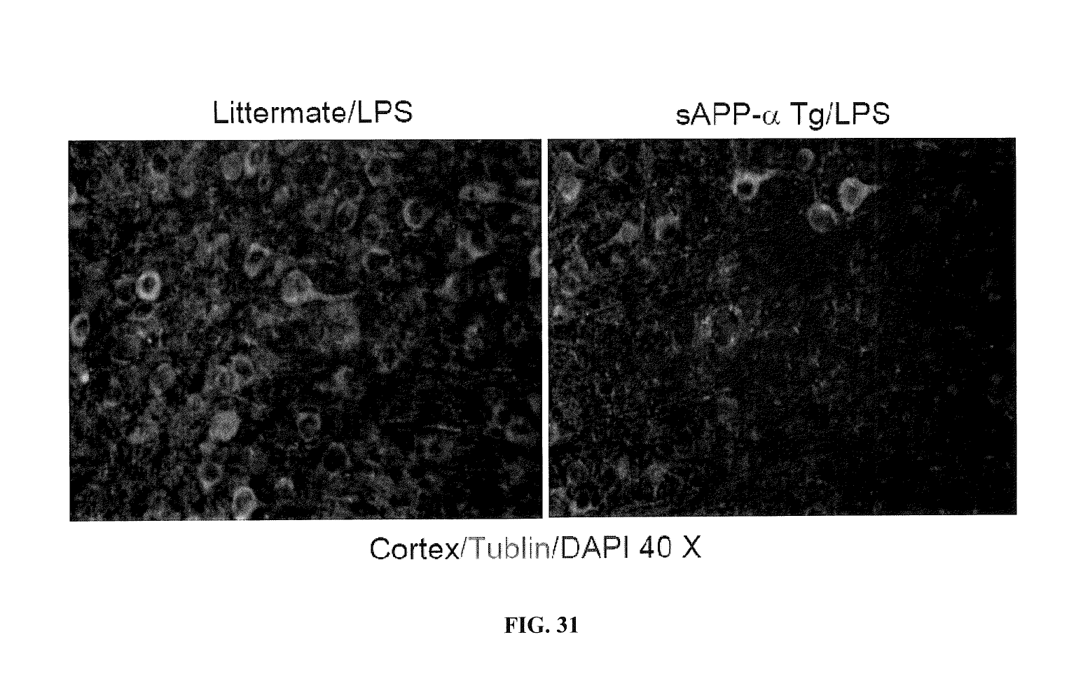

FIGS. 32A-D show that murin neurospheres treated with human sAPP-alpha exhibit GFAP-induced glial differentiation (A and B), lower levels of beta-catenin (C), and elevated levels of activated notch I (D).

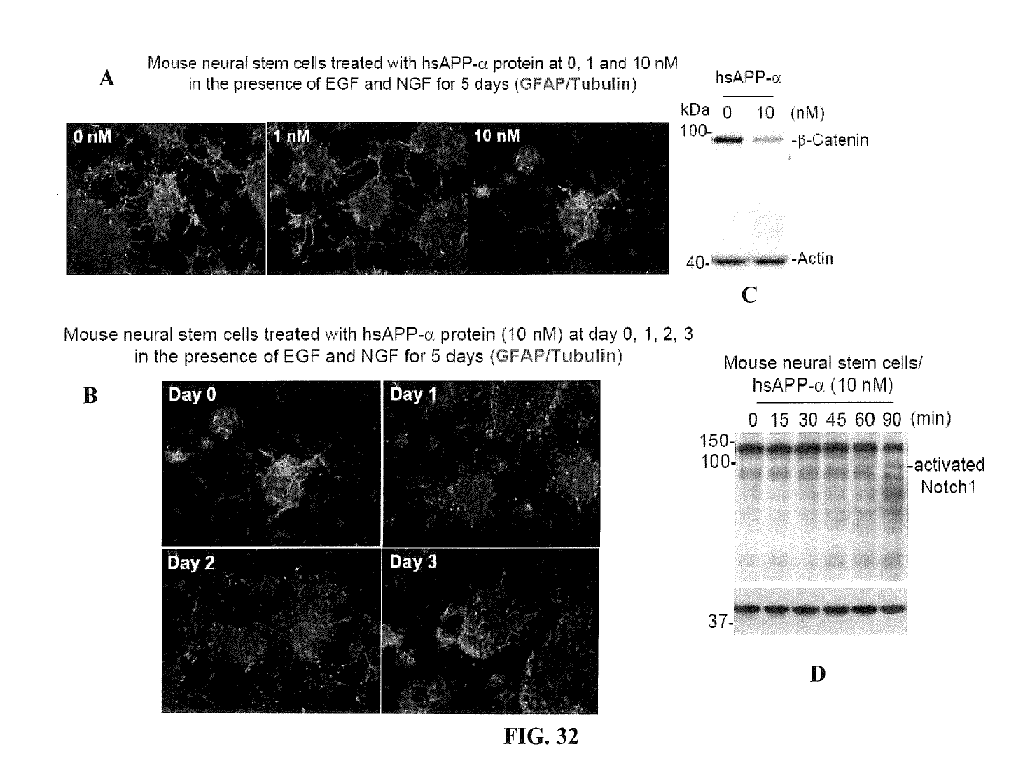

FIG. 33 shows that murine neurospheres treated with human sAPP-alpha exhibit greater GFAP-induced glial differentiation.

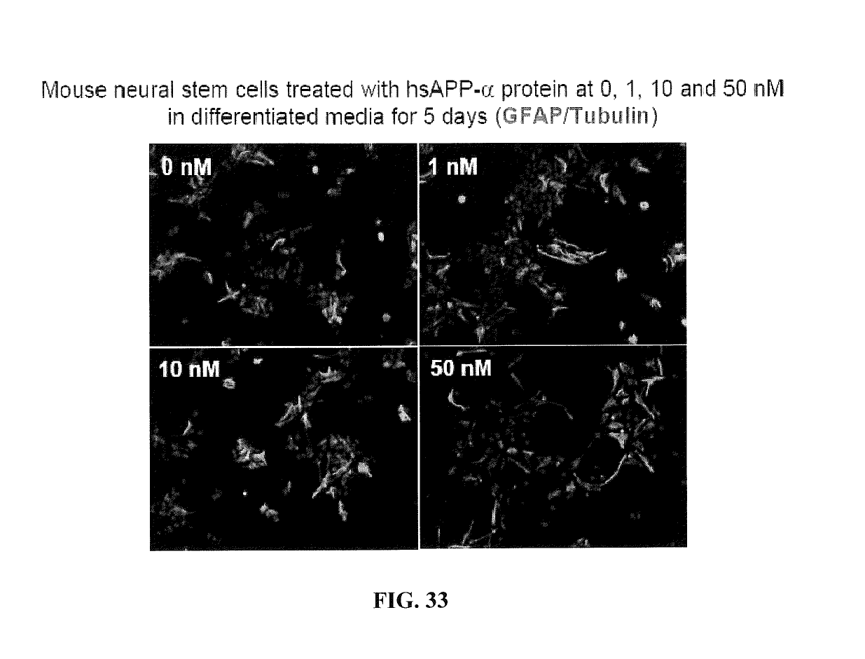

FIG. 34 shows that murine neurospheres treated IL-6 show greater GFAP-induced glial differentiation.

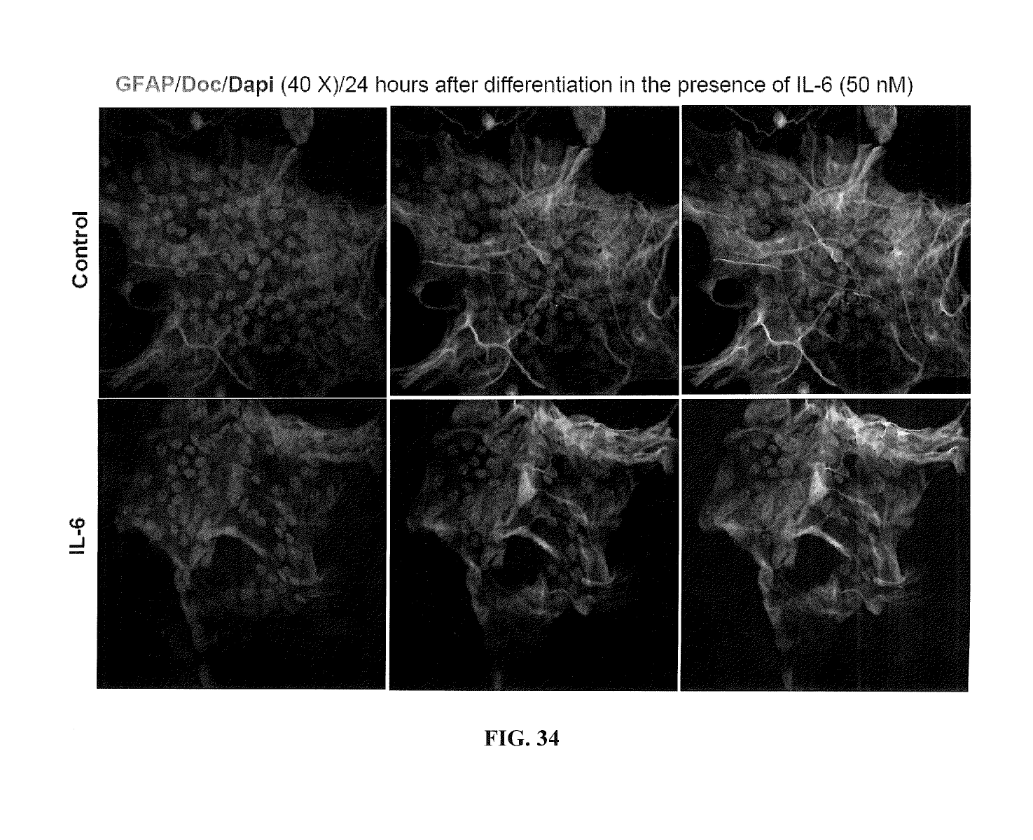

FIG. 35 illustrates sAPP-alpha plays an important role during neurodevelopment and immune system development. Elevated sAPP-alpha can cause autoimmune disorders including autism.

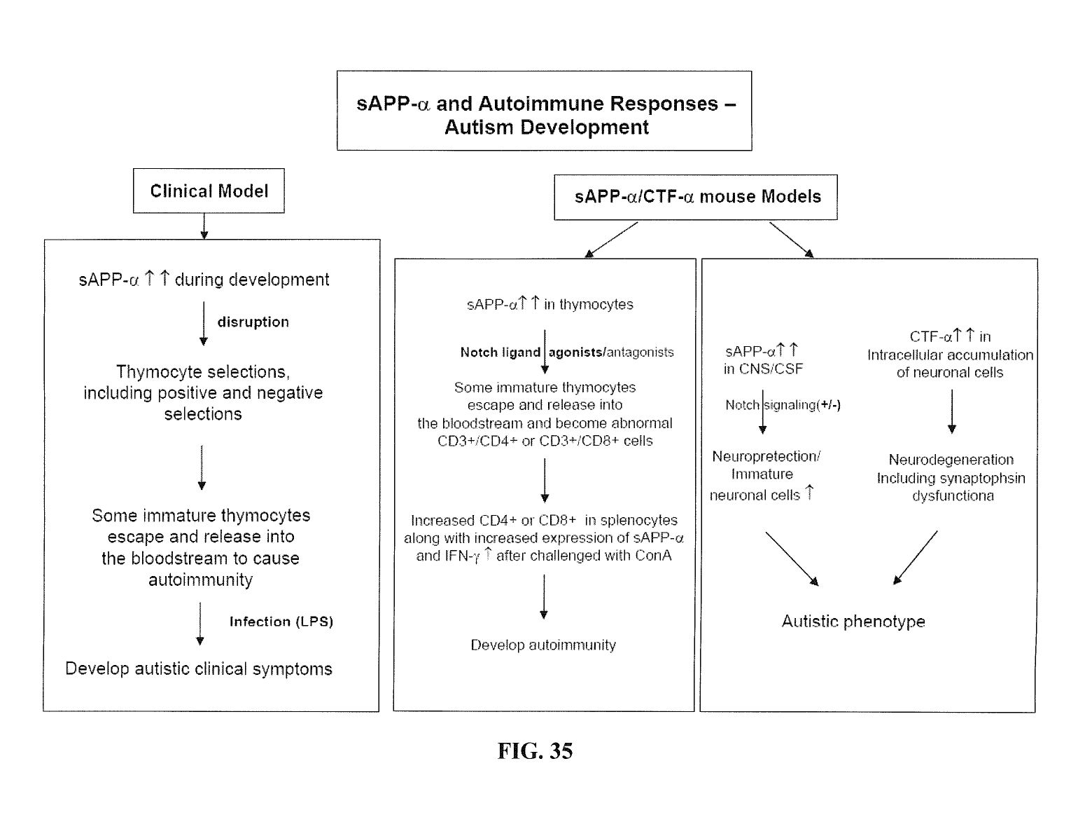

### BRIEF DESCRIPTION OF THE SEQUENCES

SEQ ID NO:1 is an amino acid sequence of human soluble amyloid precursor protein-alpha (sAPP-α) protein useful according to the present invention.

SEQ ID NO:2 is a nucleic acid sequence of human soluble amyloid precursor protein-alpha (sAPP-α) gene useful according to the present invention.

### DETAILED DISCLOSURE OF THE INVENTION

The present invention utilizes soluble amyloid precursor protein-alpha (sAPP-α) as a biomarker for prediction of a subject's risk of developing inflammatory and/or autoimmune-related disorders. In an embodiment, the present method predicts a subject's risk of developing inflammatory and/or autoimmune-related disorders associated with vaccinations. In addition, the present invention provides methods for optimizing vaccine schedules and compositions, thereby preventing, minimizing, or reducing the risks of vaccine-induced inflammatory and/or autoimmune-related disorders. Preferably, sAPP-α level of an infant is determined using methods of the present invention prior to its first administration of a vaccine composition. This allows for early detection of infants who are at risk of developing inflammatory and/or autoimmune disorders triggered by vaccinations. Accordingly, alternative vaccine schedules and/or compositions might be provided to prevent or reduce such risk.

Soluble amyloid precursor protein-alpha (sAPP-α) is generated from the non-amyloidogenic pathway in amyloid precursor protein (APP) proteolysis. APP proteolysis is a fundamental process for the production of beta-amyloid (Aβ) peptides. Aβ can be deposited as plaques in brain tissues, and thus are implicated in Alzheimer's disease (AD) pathology (Golde et al., 2000; Huse and Doms, 2000; Sambamurti et al., 2002; Funamoto et al., 2004).

Specifically, APP proteolytic products arise from the coordinated action of α-, β-, and γ-secretases. In the amyloidogenic pathway, Aβ peptides are produced by the initial action of β-secretase (BACE) cleavage, which creates an Aβ-containing C-terminal fragment (CTF) known as β-CTF or C99 (Sinha and Lieberburg, 1999; Yan et al., 1999). This proteolysis also generates an N-terminal, soluble APP-β (sAPP-β) fragment that is released extracellularly. Intracellularly, β-CTF is then cleaved by a multi-protein γ-secretase complex that results in generation of the Aβ peptide and a smaller γ-CTF, also known as C57 (De Strooper et al., 1998; Steiner et al., 1999).

Conversely, in the nonamyloidogenic pathway, APP is first cleaved at the α-secretase site, and thus results in the release of N-terminal sAPP-α. The generation of α-CTF or C83 (Hooper and Turner, 2002) is indicative of 60 -secretase activity (Hooper and Turner, 2002). Cleavage within the Aβ domain of APP results in two nonamyloidogenic pieces, and thereby prevents Aβ peptide generation from that APP (Lichtenthaler et al., 2004). Because of the limiting amount of APP in the cell and the failure to saturate the BACE pathway during APP overexpression, it is believed that the above-mentioned amyloidogenic and nonamyloidogenic pathways compete for substrate in the process of APP proteolysis (Gandhi et al., 2004). It is therefore often inferred that extracellular elevation of sAPP-α generated from nonamyloidogenic pathway activation can be taken as indirect evidence of inhibition of BACE and the associated amyloidogenic pathway, thereby providing useful information for the diagnosis and treatment of Alzheimer's disease.

It has now been discovered that elevated levels of soluble amyloid precursor protein-alpha are associated with over-reactive immune system function. For instance, individuals with elevated sAPP-α levels exhibit pro-inflammatory or inflammatory symptoms, such as altered lymphocytes profiles and increased pro-inflammatory cytokines such as IL-6 levels. Additionally, elevated serum levels of sAPP-α are present in individuals with autism.

As illustrated in the Figures, the sAPP-α overexpressing mice exhibit increased number of thymocytes, splenocytes, and glial cells. The sAPP-α overexpressing mice also exhibit increased levels of pro-inflammatory cytokines, such as IFN-γ, TNF-α, IL-6. The sAPP-α overexpressing mice also exhibit increased level of activated notch 1 expression and decreased level of beta-catenin expression in neuronal tissues.

It has also been discovered that individuals with autism also carry high levels of autoantibodies that recognize contactin-associated protein-like 2 (CASPR2, also known as CNTNAP2). CASPR2 is a neurexin that plays an important role in the neuronal adhesion and signaling processes. For example, CASPR2 is involved in axon differentiation and peripheral nervous system (PNS) development. Disruption in CASPR2 expression has been associated with social and cognitive delay and pathogenesis of autism spectrum disorders (ASD). It is now discovered that CASPR2 proteins share structural similarities with antigenic components of pertussis vaccines.

It is thus contemplated that molecular mimicry between antigenic components of pertussis vaccine compositions and CASPR2 proteins could trigger autoimmune reactions. Individuals with high sAPP-α levels are at a greater risk of developing such autoimmune over-reactions. Specifically, childhood pertussis vaccinations may induce autoimmune reactions against endogenous proteins in susceptible individuals, leading to the development and regression of iatrogenic autism in the future.

Prediction of Risk of Inflammatory and/or Autoimmune-related Disorders

In a first aspect, the present invention provides methods for predicting a subject's risk of developing inflammatory and/or autoimmune-related disorders. In an embodiment, the method comprises:

a) obtaining a biological sample from a subject;

b) measuring a level of soluble amyloid precursor protein-alpha (sAPP-α) in the sample;

c) correlating the subject's sAPP-α level to the subject's risk of developing autoimmune disorder; and

d) characterizing the subject's risk of developing autoimmune disorder.

In an embodiment, the subject's sAPP-α level is compared to a predetermined reference value, which is determined based on sAPP-α levels in a population. An elevated level of sAPP-α in the subject's biological sample, when compared to the predetermined reference value, indicates a high risk of developing autoimmune disorder.

In an embodiment, the present invention provides a method for determining individuals who are at risk of developing inflammatory and/or autoimmune-related disorders triggered by vaccinations. In an embodiment, the present invention provides a method for characterizing a subject's risk of developing vaccine-induced autoimmune disorder, comprising:

a) obtaining a biological sample from a subject that will receive vaccination, wherein the biological sample is obtained before the subject receives the vaccination;

b) measuring a level of soluble amyloid precursor protein-alpha (sAPP-α) protein in the sample;

c) correlating the subject's sAPP-α level to the subject's risk of developing vaccine-induced autoimmune disorder; and

d) characterizing the subject's risk of developing vaccine-induced autoimmune disorder.

In an embodiment, the subject's sAPP-α level is compared to a predetermined reference value, which is determined based on sAPP-α levels in a population. An elevated level of sAPP-α in the subject's biological sample, when compared to the predetermined reference value, indicates a high risk of developing vaccine-induced autoimmune disorder. In certain embodiments, the present invention characterizes the subject's risk of developing an autoimmune disorder including autism, multiple sclerosis (MS), autoimmune thyroid disease, and psoriasis. In one embodiment, the present invention characterizes the subject's risk of developing a neuronal autoimmune disorder induced by vaccination.

In certain embodiments, levels of sAPP-α are determined 15, 7, 3, 1 day(s) before, or on the same day before the subject receives vaccination. The determination can be made at multiple time points to monitor the change over time.

In a specific embodiment, levels of sAPP-α in blood samples (including plasma and/or serum) are determined using enzyme-linked immunosorbent assays (ELISA). In further embodiments, pre-, peri-, or post-natal blood samples are obtained from newly born infants or cord blood samples of the infants are collected, and the levels of sAPP-α compared to the predetermined reference values are predictive of infants' risks of developing vaccine-induced inflammatory and/or autoimmune-related disorders in later life. In a yet further embodiment, the method determines subjects who are at risk of developing autism triggered by administration of pertussis vaccine compositions.

In a preferred embodiment, sAPP-α level of an infant or child is determined, prior to administration of a vaccine composition, according to methods of the present invention. In addition, sAPP-α level may be repeatedly measured to analyze the infant or child's immune system function over time. Preferably, vaccinations are performed when the infant or child is at a lower risk of developing inflammatory and/or autoimmune-related disorders. Furthermore, the present method can also detect a subject that is suffering from symptoms of inflammatory and/or autoimmune-related disorders, and thus allows for avoidance of worsening of the symptoms.

The term “subject,” as used herein, describes an organism, including mammals such as primates. Mammalian species that can benefit from the subject methods include, but are not limited to, apes, chimpanzees, orangutans, humans, monkeys; and domesticated and/or laboratory animals such as dogs, cats, horses, cattle, pigs, sheep, goats, chickens, mice, rats, guinea pigs, and hamsters. Typically, the subject is a human. In one embodiment, the subject is an infant of 0 to 12 months of age, or an infant of 0 to 24 months of age. In another embodiment, the subject is a child of 2 to 17 years of age, or a child of 2 to 12 years of age.

The term “biological sample,” as used herein, includes but is not limited to a sample containing tissues, cells, and/or biological fluids isolated from a subject. Examples of biological samples include but, are not limited to, tissues, cells, biopsies, blood, lymph, serum, plasma, urine, saliva, and tears. In various embodiments, biological samples are obtained from, or derived from, blood, including plasma, serum, and blood cells. In a specific embodiment, blood samples are obtained from, or derived from, cord blood, prenatal, perinatal, and/or postnatal blood of a subject. In addition, one skilled in the art would realize that some samples would be more readily analyzed following a fractionation or purification procedure, for example, separation of whole blood into serum or plasma components.

The predetermined reference value can be established by skilled healthcare practitioners. For instance, the predetermined reference value can be established by measuring the levels of the biomarker in a normal population sample and correlating such levels with factors such as the incidence, severity, and/or frequency of developing inflammatory and/or autoimmune-related disorders in a population. Such population is naturally composed of subjects with varying degrees of immune system function and risks of developing inflammatory and/or autoimmune-related disorders. Thus, a subject's biomarker level as compared against the corresponding reference biomarker value correlates to the subject's risk of inflammatory and/or autoimmune disorders. In addition, the predetermined value can be a single value, multiple values, a single range, or multiple ranges. Thus, a subject's risk may be predicted by determining in which of the predetermined reference ranges the subject's level falls. Alternatively, the relative level of risk of inflammatory and/or autoimmune disorders can be determined based upon the alteration of a subject's biomarker level as compared against the corresponding biomarker levels of a population. Further, the predetermined reference value is preferably provided by using the same assay technique as is used for measurement of the subject's biomarker level, to avoid any error in standardization.

The term “sAPP-α biomarker,” as used herein, includes the mature full length human sAPP-α peptide generated by cleavage of the amyloid precursor protein by α-secretase, and fragments thereof identifiable as originating from sAPP-α. In an embodiment, the human sAPP-α peptide has an amino acid sequence of SEQ ID NO: 1. In certain embodiments, the human sAPP-α peptide has at least 80%, 85%, 90%, 93%, 95%, 97%, 98%, or 99% sequence identity to SEQ ID NO: 1.

In an embodiment, the present invention provides methods for predicting a subject's risk of developing inflammatory and/or autoimmune disorders that are triggered by, or associated with, exposures to non-native antigens. Such antigens include, but are not limited to, virus, bacteria, fungi, pathogens, allergens, microorganisms, insects, cells or cellular components, and molecules such as proteins, peptides, nucleic acid molecules, polynucleotides, carbohydrates, lipids, glycolipids, and lipid peptides.

In a specific embodiment, the subject is exposed to non-native antigens during or as a result of receiving a medical treatment or procedure. Such exposures are usually through administration of therapeutic agents, such as administration of chemical compounds, proteins and peptides such as interferons and antibodies, nucleic acid molecules, polynucleotides, carbohydrates, lipids, glycolipids, and lipid peptides, to the subject.

In a specific embodiment, such medical treatment is an active or passive immunization of a subject against a disease or infection. Such immunization agents include, but are not limited to, agents against pertussis, polio, hepatitis (e.g. hepatitis A and hepatitis B), measles, mumps, rubella, influenza, smallpox, zoster, anthrax, tetanus, rotavirus, rabies, pneumonia, chickenpox, meningococcus, diphtheria, anpapillomavirus, anthrax, plague, encephalitis, pneumococcus, pneumonia, typhus, typhoid fever, streptococcus, staphylococcus, neisseria, Lyme disease, cholera, E. coli, shigella, leishmania, leprosy, cytomegalovirus (CMV), respiratory syncytial virus, Epstein Barr virus, herpes, parainfluenza, adenovirus, human immunodeficiency virus (HIV), varicella, yellow fever, flavivirus, dengue toxoplasmosis, coccidiomycosis, schistosomiasis, and malaria.

In an embodiment, the present invention characterizes a subject's risk of developing vaccine induced autoimmune disorder, if the subject is administered to a vaccine composition comprising an agent that can induce autoimmunity. Examples of agents that can induce autoimmunity include, but are not limited to, antigenic peptides having a sequence similar to the CASPR2/CNTNP2 peptide and mercury from thimerosal-containing vaccines. In certain embodiments, agents that can induce autoimmunity are antigenic peptides having identical or similar (such as having at least 90%, 93%, 95%, 97%, 98%, or 99% sequence identity) amino acid sequences to a native peptide sequence of the subject. In certain embodiments, agents that can induce autoimmunity are antigenic peptides that bind specifically to an autoantibody of the subject.

In one embodiment, a longitudinal analysis of the subject's immune function is performed, including determinig sAPP-alpha level, analyzing white blood cell populations, analyzing phenotypes of CD4+ vs. CD8+ T-cells, and de termining T-cell and/or B-cell populations, for determining whether the subject has immune derangement and the extent of such immune derangement. Such determination can be made using methods known in the art, such as flow cytometry, Western blot, ELISA, and immunochemistry staining.

“Specific binding” or “specificity” refers to the ability of an antibody or other agent to exclusively bind to an epitope presented on an antigen while having relatively little non-specific affinity with other proteins or peptides. Specificity can be relatively determined by binding or competitive binding assays, using, e.g., Biacore instruments. Specificity can be mathematically calculated by, e.g., an about 10:1, about 20:1, about 50:1, about 100:1, 10.000:1 or greater ratio of affinity/avidity in binding to the specific antigen versus nonspecific binding to other irrelevant molecules.

In an embodiment, the method of the present invention further involves determination of the level of a second biomarker that is associated with immune function and/or inflammation. The biomarkers of the invention include molecules, genes, proteins, cellular components, and variants or fragments thereof A biomarker protein comprises the entire or partial amino acid sequence of interest. A biomarker nucleic acid includes DNA that encodes the entire or partial amino acid sequence of the protein or peptide of interest, or encodes proteins or peptides that are involved in the expression, secretion and/or transport of the protein or peptide of interest. Such DNA biomarkers include DNA comprising the entire or partial sequence of the nucleic acid sequence encoding the biomarker protein or peptide, or the complement of such a sequence. The biomarker nucleic acids also include RNA comprising the entire or partial sequence of any of the nucleic acid sequences of interest. Biomarkers of the present invention also include molecules whose production is altered under pro-inflammatory or inflammatory conditions.

Exemplified biomarkers of the present invention include, for example, cytokines including tumor necrosis factors such as TNF-α and TNF-β; lymphocyte activating factor (LAF), B-cell stimulating factor (BSF-1), interferons such as Interferon-alpha (IFN-α), Interferon-beta (IFN-β), Interferon-gamma (IFN-γ); tissue growth factor (TGF) β; the interleukin family such as Interleukin-1 (IL-1), Interleukin-2 (IL-2), Interleukin-3 (IL-3), Interleukin-4 (IL-4), Interleukin-5 (IL-5), Interleukin-6 (IL-6), Interleukin-7 (IL-7), Interleukin-8 (IL-8), Interleukin-9 (IL-9), Interleukin-10 (IL-10), Interleukin-11 (IL-11), Interleukin-12 (IL-12), Interleukin-13 (IL-13), Interleukin-14 (IL-14), Interleukin-15 (IL-15), Interleukin-16 (IL-16), Interleukin-17 (IL-17), Interleukin-18 (IL-18), Interleukin-19 (IL-19), Interleukin-20 (IL-20), Interleukin-21 (IL-21), Interleukin-22 (IL-22), Interleukin-23 (IL-23), Interleukin-24 (IL-24), Interleukin-25 (IL-25), Interleukin-26 (IL-26), Interleukin-27 (IL-27), Interleukin-28 (IL-28), Interleukin-29 (IL-29), Interleukin-30 (IL-30), Interleukin-31 (IL-31), Interleukin-32 (IL-32), Interleukin-33 (IL-33), Interleukin-34 (IL-34), Interleukin-35 (IL-35); the interleukin receptor family; the macrophage inflammatory protein family such as macrophage inflammatory protein 2 (MIP-2) and macrophage inflammatory protein 1α (MIP-1α); macrophage colony-stimulating factor (M-CSF); monocyte chemotactic protein-1 (MCP-1); nitric oxide (NO) and nitric oxide synthases; and immunoglobulins such as IgA, IgG, IgM, IgD, and IgE.

Immunoglobulins include IgG, IgM, IgD, IgE, IgA and subtypes such as for example IgG1, IgG2, IgG3, IgG4, IgA1, and IgA2. They further include molecules in monomeric or multimeric form, whether digested from whole antibody or produced by other means.

In certain embodiments, a second biomarker includes, but is not limited to, IFN-γ, TNF-α, IL-6, activated notch 1, beta-catenin, activated (cleaved) version of caspase 3, MAP2, NeuN, Iba1, and GFAP. In certain embodiments, the numbers of thymocytes, splenocytes, glial cells, T cells, and/or B cells are measured to determine the subject's immune function.

Exemplified biomarkers of the present invention further include autoantibodies, autoreactive lymphocytes, and cellular substances that indicate destruction of internal cellular components, tissues or organs. For example, the biomarkers may include cell surface antigens, including but not limited to, CD4, CD8, CD154, LFA-1, CD80, CD86 and ICAM-1. Preferably, the levels of biomarkers of the present invention indicate the extent of immunological over-reaction.

Furthermore, the populations and cellular profiles of various white blood cells may be determined for characterization a subject's immune conditions and prediction of risks of immune disorders. For example, the population and cellular profiles of neutrophils, eosinophils, basophils, monocytes, macrophages, lymphocytes, and dendritic cells may be determined. In addition, the expression level and phenotypes of various proteins, peptides, glycoproteins and lipids, which are expressed in or by various white blood cells or their receptors, may be determined.

In an embodiment, sAPP-α level is analyzed in combination with a second biomarker. In other embodiments, the levels of a plurality of biomarkers are determined. The combinations of sAPP-α level and the plurality of biomarker levels are used to predict a subject's risk of developing inflammatory and/or autoimmune-related disorders.

Additionally or alternatively, the ratio of various biomarker levels of interest is determined for analysis in combination with sAPP-α level. The determination and analysis of the levels of sAPP-α and one or more biomarkers may be carried out separately or simultaneously. Several biomarkers may be combined into one test for efficient processing of multiple samples from a subject.

In a specific embodiment, sAPP-α level is analyzed in combination with the population of T lymphocytes. In a further specific embodiment, sAPP-α level is analyzed in combination with the level of cluster of differentiation 4 (CD4), which is a glycoprotein expressed on the surface of T helper cells, regulatory T cells, monocytes, macrophages, and dendritic cells. Alternatively, sAPP-α level may be analyzed in combination with the level of cluster of differentiation 8 (CD8), which is a transmembrane glycoprotein that serves as a co-receptor for the T cell receptor (TCR). Additionally, sAPP-α level may be analyzed in combination with the ratio of CD4 vs. CD8.

Specifically, for prediction of risk of drug-induced inflammatory and/or autoimmune-related disorders, a biological sample may be assayed to determine the presence and/or level of an endogenous molecule (e.g. a protein, a peptide) has cross-reactivity with an antigenic component or an etitope thereof of the vaccine composition. In addition, the biological sample may be assayed to determine the presence and/or level of antibodies that specifically bind to an antigenic component or an etitope thereof of the vaccine composition. Furthermore, genomic DNA may be sequenced to determine DNA that encodes such endogenous molecule. Such information is analyzed in combination with sAPP-α level of a subject for more accurate prediction of the subject's risk for developing drug-induced inflammatory and/or autoimmune-related disorders.

Antibodies that immunospecifically bind to an antigenic component of the vaccine composition can be identified, for example, by immunoassays, Western blot, BIAcore, radioimmunoassays (RIA) and enzyme-linked immunosorbent assays (ELISAs), or other techniques known to those of skill in the art.

In an embodiment, the biological sample is assayed to determine the presence and/or level of an endogenous protein or peptide of interest. Such protein or peptide exhibits between about 70% to about 100%, or preferably at least 75%, or at least 80%, or at least 90% sequence homology with the amino acid sequence of an antigenic component of the vaccine composition. Alternatively, genomic DNA may be sequenced to determine DNA that encodes such endogenous protein or peptide.

A further embodiment contemplates longitudinal analysis of sAPP-α levels over time for prediction of the subject's risk. Specifically, the levels of sAPP-α and/or other biomarkers are determined multiple times over time to monitor the change of a subject's conditions. Such testing of multiple samples allows for the identification of changes in the biomarker level over time. Increases or decreases in the level of biomarker(s), as well as the absence of change in levels, would provide useful information about the subject's immune system.

### Customization of Vaccine Schedules and Compositions

Another aspect of the invention provides methods for optimizing vaccine schedules and compositions for preventing or reducing risks of vaccine-induced inflammatory and/or autoimmune-related disorders. In an embodiment, the method comprises:

a) obtaining a biological sample from a subject;

b) measuring a level of soluble amyloid precursor protein-alpha (sAPP-α) in the sample;

c) correlating the subject's sAPP-α level to the subject's risk of developing autoimmune related disorder; and

d) administering a customized vaccine schedule to a subject if the subject's sAPP-α level correlates to a high risk of developing vaccine-induced autoimmune disorder.

In certain embodiments, levels of sAPP-α are determined 15, 7, 3, 1 day(s) before, or on the same day before the subject receives vaccination. The determination can be made at multiple time points to monitor the change over time.

In one embodiment, a customized vaccine is provided to a subject with a high risk of developing vaccine-induced autoimmune disorder. In certain embodiments, the high-risk subject receives a customized vaccine composition comprising a reduced amount of pro-inflammatory adjuvant, and/or receives an anti-inflammatory or immune-suppressive agent before, together with, or after the vaccine composition. In one embodiment, the high-risk subject's sAPP-α level is measured at a later time point, or at multiple time points over time. If, at a time point, the subject's sAPP-α level correlates to a low risk of developing vaccine-induced autoimmune disorder, a vaccine composition is administered. In another embodiment, the high-risk subject receives a customized vaccine composition that does not comprise an agent that induces autoimmunity.

The present invention also contemplates methods for providing customized vaccine schedules or compositions for individuals having certain risks of developing inflammatory and/or autoimmune-related disorders. A vaccine schedule is a program that includes the timing, doses and routes of administration of a vaccine composition. A vaccine composition is an antigenic preparation that comprises one or more immunogenic antigens used to produce active immunity to a disease. Such compositions may contain suitable pharmaceutically acceptable carriers, such as excipients, adjuvants and/or auxiliaries, and other therapeutically inactive ingredients.

In an embodiment, the timing of vaccine administration can be optimized by considering the subject's risk of developing inflammatory and/or autoimmune disorders. Preferably, a subject is immunized when its sAPP-α level correlates to a low risk of developing inflammatory and/or autoimmune-related disorders. In addition, sAPP-α levels may be repeatedly measured to monitor the changes of the subject's immune system function. Vaccinations may be postponed within an acceptable timeframe until the sAPP-α level falls back to a range that correlates to a low risk level. Furthermore, for subjects whose sAPP-α levels indicate certain risks of developing inflammatory and/or autoimmune-related disorders, vaccines may be administered within a recommended or mandatory timeframe when the subject's sAPP-α is at a lower level.

In addition, vaccine schedules can be customized by adjusting and/or reducing the dosage of one or more immunogens and/or therapeutically inactive immunogenic ingredients. A lower dosage might be administered to a subject with an over-reactive immune system and having a higher risk of developing inflammatory and/or autoimmune-related disorders. In addition, the dosage of immunogenic ingredients may be reduced. Further, immunosuppressive agents may be administered independently or in combination with the vaccine composition.

In addition, a vaccine composition may be customized to eliminate or reduce the risk of autoimmune responses, especially for individuals with an elevated sAPP-α level and/or over-reactive immune system function. In an embodiment, a vaccine composition may be customized by selecting an immunogen that does not contain such antigenic component. In addition, a vaccine composition may be customized by eliminating such antigenic component. Further, a vaccine composition may be customized by reducing the amino acid sequence homology between the antigenic component and an endogenous molecule.

Furthermore, for individuals who are at risk of developing inflammatory and/or autoimmune disorders, a degree of amino acid sequence similarity between an antigenic component of the vaccine composition and an endogenous peptide or a fragment thereof may be determined. Consequently, the vaccine compositions may be customized by reducing the dosage of the antigenic molecule, removing the antigenic molecule from the vaccine composition, or substituting an antigenic etitope with another etitope that has a lesser sequence homology with the endogenous peptide or a fragment thereof.

In a specific embodiment, vaccine schedules and compositions are optimized based upon the levels of sAPP-α and/or biomarkers associated with immune system function. In addition, flow cytometry analysis of immune cell populations can be performed to determine a subject's immune system function. In this way, individuals with symptoms of immune derangement can be detected before vaccination. Further, the extent of derangement can be determined to provide useful information for customized selection and dosing of immunogens. For instance, individuals with moderate to severe immune derangements may be immunized using a reduced dosage. Alternatively, vaccine compositions may be adjusted by eliminating highly immunogenic adjuvants or administering less-immunogenic compositions. In a specific embodiment, the method determines subjects who are at risk of developing autism triggered by administration of pertussis vaccine compositions, and provides customized pertussis vaccination schedules and compositions to prevent or reduce such risks.

The methods of the present invention are useful for predicting, preventing, minimizing, and/or reducing a subject's risk of developing inflammatory and/or autoimmune-related disorders in various life stages including during infancy, childhood, adolescence, and adulthood. Usually, the first administration of a vaccine composition occurs within the first 180 days from the birth. Subsequent “catch-up vaccines” may be performed during childhood and/or adolescence. Therefore, customized vaccine schedules and compositions of the present invention advantageously prevent or reduce risks of infants and children for developing inflammatory and/or autoimmune-related disorders in later life. Furthermore, the present method detects infants and children with existing symptoms of inflammatory and autoimmune-related disorders, and provides for customized vaccine schedules and compositions to prevent or minimize worsening of the diseases.

Inflammatory and/or Autoimmune-related Disorders

Autoimmune-related disorders are characterized by an attack of the immune system against its own body's tissues. The methods of the present invention are useful for predicting, preventing, minimizing, and/or reducing risks for inflammatory and/or autoimmune-related disorders, including but not limited to, autism, multiple sclerosis (MS), autoimmune thyroid disease, psoriasis, Guillain-Barré syndrome, systemic lupus erythematosus, postvaccinal encephalomyelitis, seizure, paralysis, mental retardation, attention-deficit hyperactivity disorder (ADHD), and dyslexia.

In addition, the methods of the present invention are useful for predicting, preventing, minimizing, and/or reducing risks of developing inflammatory and/or autoimmune-related disorders, including but not limited to, scleroderma, autoimmune hepatitis, diabetes mellitus, ulcerative colitis, Myasthenia gravis, systemic lupus erythematosus, Graves' disease, idiopathic thrombocytopenia purpura, hemolytic anemia, multiple myositis/dermatomyositis, Hashimoto's disease, autoimmune hypocytosis, Sjogren's syndrome, angitis syndrome and drug-induced autoimmune-related disorders (e.g., drug-induced lupus), particularly vaccine-induced autoimmune diseases.

In addition, the methods of the present invention are useful for predicting, preventing, minimizing, and/or reducing risks of developing inflammatory and/or autoimmune-related disorders, including but not limited to, Hashimoto's disease, thyroiditis, IgA nephropathy, gastritis, adrenalitis (Addison's), ovaritis, myasthenia gravis, gonadal failure, hypoparathyroidism, alopecia, malabsorption syndrome, pernicious anemia, hepatitis, anti-receptor antibody diseases, schizophrenia, Idiopathic thrombocytopenic purpura, Alzheimer's disease, narcolepsy, pernicious anaemia, depression, hypopituitarism, diabetes insipidus, sicca syndrome, systemic lupus erythematous or Lupus, scleroderma, polymyositis, inflammatory bowel disease, dermatomyositis, ulcerative colitis, Crohn's disease, vasculitis, psoriatic arthritis, exfoliative psoriatic dermatitis, vasculitis, pemphigus vulgaris, Sjorgren's syndrome, uvoretinitis, glomerulonephritis, post myocardial infarction cardiotomy syndrome, pulmonary hemosiderosis, amyloidosis, sarcoidosis, and aphthous stomatitis.

Furthermore, the methods of the present invention are useful for predicting, preventing, minimizing, and/or reducing risks of developing inflammatory and/or autoimmune-related disorders included by vaccinations against diseases, including but not limited to, pertussis, polio, hepatitis (e.g. hepatitis A and hepatitis B), measles, mumps, rubella, influenza, smallpox, zoster, anthrax, tetanus, rotavirus, rabies, pneumonia, chickenpox, meningococcus, diphtheria, anpapillomavirus, anthrax, plague, encephalitis, pneumococcus, pneumonia, typhus, typhoid fever, streptococcus, staphylococcus, neisseria, lyme disease, cholera, E. coli, shigella, leishmania, leprosy, cytomegalovirus (CMV), respiratory syncytial virus, Epstein Barr virus, herpes, parainfluenza, adenovirus, human immunodeficiency virus (HIV), varicella, yellow fever, flavivirus, dengue toxoplasmosis, coccidiomycosis, schistosomiasis, and malaria.

Determination of Presence and/or Levels of Biomarkers

The sAPP-α biomarker and biomarkers associated with immune function and/or inflammation can be determined by quantitative immunological detection methods, such as for example, enzyme-linked immunosorbant assays (ELISA), Western blot, immunological assays, microarrays and radioimmunoassays. In addition, immune cell populations and profiles are routinely examined using flow cytometry analysis.

Specifically, methods for detecting biomarkers of the invention comprise any methods that determine the quantity or the presence of the biomarkers either at the nucleic acid or protein level. Such methods are well known in the art, and include, but are not limited to, Western blots, Northern blots, Southern blots, ELISA, immunoprecipitation, immunofluorescence, radioimmunoassay, flow cytometry, immunocytochemistry, nucleic acid hybridization techniques, nucleic acid reverse transcription methods, and nucleic acid amplification methods. In particular embodiments, overexpression of a biomarker is detected on a protein level using, for example, antibodies that are directed against specific biomarker proteins. These antibodies can be used in various methods such as Western blot, ELISA, immunoprecipitation, or immunocytochemistry techniques.

As is known in the art, polypeptides or proteins in test samples are commonly detected with immunoassay devices and methods. Alternatively, or additionally, aptamers can be selected and used for binding of even greater specificity, as is well known in the art. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule.

Preferably, the biomarkers are analyzed using an immunoassay, although other methods are well known to those skilled in the art (for example, the measurement of biomarker RNA levels). The presence or amount of a biomarker is generally determined using antibodies specific for each biomarker and detecting specific binding. Any suitable immunoassay may be utilized, for example, enzyme-linked immunoassays (ELISA), radioimmunoassays (RIAs), competitive binding assays, and the like. Specific immunological binding of the antibody to the biomarker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the biomarkers is also contemplated by the present invention and is well known by one of ordinary skill in the art. The antibodies can be immobilized onto a variety of solid supports, such as magnetic or chromatographic matrix particles, the surface of an assay place (such as microtiter wells), pieces of a solid substrate material (such as plastic, nylon, paper), and the like. An assay strip can be prepared by coating the antibody or a plurality of antibodies in an array on solid support. This strip can then be dipped into the test sample and then processed quickly through washes and detection steps to generate a measurable signal, such as a colored spot.

The analysis of a plurality of biomarkers may be carried out separately or simultaneously with one test sample. Several biomarkers may be combined into one test for efficient processing of a multiple of samples. In addition, one skilled in the art would recognize the value of testing multiple samples (for example, at successive time points) from the same individual. Such testing of serial samples will allow the identification of changes in biomarker levels over time. Increases or decreases in biomarker levels, as well as the absence of change in biomarker levels, would provide useful information about the disease status that includes, but is not limited to identifying the approximate time from onset of the event, the presence and amount of salvageable tissue, the appropriateness of drug therapies, the effectiveness of various therapies, identification of the severity of the event, identification of the disease severity, and identification of the patient's outcome, including risk of future events.

An assay consisting of a combination of the biomarkers referenced in the instant invention may be constructed to provide relevant information related to differential diagnosis. Such a panel may be constructed using 1, 2, 3, 4, 5, 6, 7, 8, 9, 10, 15, 20, or more or individual biomarkers, though a number lower than 4 biomarkers is the most preferred embodiment. The analysis of a single biomarker or subsets of biomarkers from a larger panel of biomarkers can be carried out in accord with methods described within the instant invention to optimize clinical sensitivity or specificity in various clinical settings. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W. B. Saunders and Company, p. 496).

The analysis of biomarkers can be carried out in a variety of physical formats as well. For example, the use of microtiter plates or automation can be used to facilitate the processing of large numbers of test samples. Alternatively, single sample formats can be developed to facilitate immediate treatment and diagnosis in a timely fashion, for example, in ambulatory transport or emergency room settings. Particularly useful physical formats comprise surfaces having a plurality of discrete, addressable locations for the detection of a plurality of different analytes. Such formats include protein microarrays, or “protein chips” (see, e.g., Ng and Ilag, J. Cell Mol. Med. 6: 329-340 (2002)) and capillary devices.

In another embodiment, the present invention provides a kit for the analysis of biomarkers. Such a kit preferably comprises devices and reagents for the analysis of at least one test sample and instructions for performing the assay. The kit may contain aptamers specific for a target biomarker. Optionally the kits may contain one or more means for using information obtained from immunoassays performed for a biomarker panel to rule in or out certain diagnoses. Biomarker antibodies or antigens may be incorporated into immunoassay diagnostic kits depending upon which biomarker autoantibodies or antigens are being measured. A first container may include a composition comprising an antigen or antibody preparation. Both antibody and antigen preparations should preferably be provided in a suitable titrated form, with antigen concentrations and/or antibody titers given for easy reference in quantitative applications.

The kits may also include an immunodetection reagent or label for the detection of specific immunoreaction between the provided antigen and/or antibody, as the case may be, and the diagnostic sample. Suitable detection reagents are well known in the art as exemplified by radioactive, enzymatic or otherwise chromogenic ligands, which are typically employed in association with the antigen and/or antibody, or in association with a second antibody having specificity for first antibody. Thus, the reaction is detected or quantified by means of detecting or quantifying the label. Immunodetection reagents and processes suitable for application in connection with the novel methods of the present invention are generally well known in the art.

The reagents may also include ancillary agents such as buffering agents and protein stabilizing agents, e.g., polysaccharides and the like. The kit may further include where necessary agents for reducing background interference in a test, agents for increasing signal, software and algorithms for combining and interpolating biomarker values to produce a prediction of clinical outcome of interest, apparatus for conducting a test, calibration curves and charts, standardization curves and charts, and the like.

The measurement of the concentration of the biomarker in the biological sample may employ any suitable the biomarker antibody or aptamer to detect the protein. Such aptamers or antibodies may be presently extant in the art or presently used commercially, or may be developed by techniques now common in the field of immunology.

As used herein, the term “antibody” refers to an intact immunoglobulin having two light and two heavy chains or any antibody fragments thereof sufficient to bind a target of interest. Thus a single isolated antibody or antibody fragment may be a polyclonal antibody, a high affinity polyclonal antibody, a monoclonal antibody, a synthetic antibody, a recombinant antibody, a chimeric antibody, a humanized antibody, or a human antibody.

The term “antibody fragment,” as used herein, refers to less than an intact antibody structure, including, without limitation, an isolated single antibody chain, an Fv construct, a Fab construct, a light chain variable or complementarity determining region (CDR) sequence, etc. A recombinant molecule bearing the binding portion of an antibody, e.g., carrying one or more variable chain CDR sequences that bind the biomarker, may also be used in a diagnostic assay of this invention.

As used herein, the term “antibody” may also refer, where appropriate, to a mixture of different antibodies or antibody fragments that bind to the biomarker. Such different antibodies may bind to a different portion of the biomarker than the other antibodies in the mixture. Such differences in antibodies used in the assay may be reflected in the CDR sequences of the variable regions of the antibodies. Such differences may also be generated by the antibody backbone, for example, if the antibody itself is a non-human antibody containing a human CDR sequence, or a chimeric antibody or some other recombinant antibody fragment containing sequences from a non-human source. Antibodies or fragments useful in the method of this invention may be generated synthetically or recombinantly, using conventional techniques or may be isolated and purified from plasma or further manipulated to increase the binding affinity thereof.

Similarly, the antibodies may be tagged or labeled with reagents capable of providing a detectable signal, depending upon the assay format Such labels are capable, alone or in concert with other compositions or compounds, of providing a detectable signal. Where more than one antibody is employed in a diagnostic method, the labels are desirably interactive to produce a detectable signal. Most desirably, the label is detectable visually, e.g. calorimetrically. A variety of enzyme systems operate to reveal a calorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting biomarker-antibody complex in applicable assays. Still other labels include fluorescent compounds, radioactive compounds or elements. Preferably, an antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

Detectable labels for attachment to antibodies useful in diagnostic assays of this invention may be easily selected from among numerous compositions known and readily available to one skilled in the art of diagnostic assays. The anti-body aptamers, antibodies, or fragments useful in this invention are not limited by the particular detectable label or label system employed. Thus, selection and/or generation of suitable antibodies and aptamers with optional labels for use in this invention is within the skill of the art, provided with this specification, the documents incorporated herein, and the conventional teachings of immunology.

Similarly the particular assay format used to measure the biomarker in a biological sample may be selected from among a wide range of immunoassays, such as enzyme-linked immunoassays, such as those described in the examples below, sandwich immunoassays, homogeneous assays, or other assay conventional assay formats. One of skill in the art may readily select from any number of conventional immunoassay formats to perform this invention.

Other reagents for the detection of protein in biological samples, such as peptide mimetics, synthetic chemical compounds capable of detecting the biomarker may be used in other assay formats for the quantitative detection in biological samples, such as Western blots, flow cytometry, etc.

Following are examples that illustrate procedures for practicing the invention. These examples should not be construed as limiting. All percentages are by weight and all solvent mixture proportions are by volume unless otherwise noted.

### EXAMPLE 1

### Identification of sAPP-α as a Biomarker for Prediction of Risk of Autoimmune Disorders

This Example reveals that elevated sAPP-α levels indicate abnormal immune function and increased risks of developing vaccine-induced autoimmune diseases. Specifically, immune tissues of 3-month-old mice were transgenically modified to produce high levels of sAPP-α. The T-cell, B-cell and spleen cell profiles of the mice were assayed to examine changes in immune responses due to increased levels of sAPP-α. The results show that mice with elevated sAPP-α levels exhibit dramatically altered T-cell and B-cell profiles, especially within thymus and spleen tissues. Additionally, spleen cells derived from mice with high levels of sAPP-α exhibit symptoms of exaggerated responses to immune stimulation.

In addition, it is shown that mice having abnormal immune function associated with elevated levels of sAPP-α more frequently develop antigen-induced autoimmune disorders. Specifically, mice with elevated levels of sAPP-α are immunized with antigenic components of pertussis vaccine that share amino acid sequence homology with CASPR2. The results show that such mice exhibit significantly high levels of IL-6 in the central nervous system post-immunization, as compared to mice with normal sAPP-α levels. In addition, these mice develop a variety of cognitive and behavioral abnormalities that are present in autism, such as social isolation. Thus, sAPP-α is a useful biomarker for detection of immune dysfunction and prediction of risks of developing autoimmune diseases, particularly those diseases induced by vaccinations.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A method of characterizing a subject's risk of developing vaccine-induced autoimmune disorder, comprising:
a) obtaining a biological sample from a subject who is a candidate for vaccination, wherein the biological sample is obtained before the subject receives the vaccination;
b) measuring a level of soluble amyloid precursor protein-alpha (sAPP-α) protein in the sample;
c) correlating the subject's sAPP-α level to the subject's risk of developing vaccine-induced autoimmune disorder; and
d) characterizing the subject's risk of developing vaccine-induced autoimmune disorder.

2. The method, according to claim 1, wherein step (c) comprises comparing the subject's sAPP-α level to a predetermined reference value, wherein the predetermined reference value is determined based on sAPP-α levels in a population, and wherein an elevated level of sAPP-α in the subject's biological sample, when compared to the predetermined reference value, indicates a higher than normal risk of developing vaccine-induced autoimmune disorder.

3. The method, according to claim 1, wherein the subject is a child or an infant.

4. The method, according to claim 1, wherein the biological sample is a blood sample.

5. The method, according to claim 4, wherein the biological sample is selected from a cord blood, prenatal blood, perinatal blood, or postnatal blood sample.

6. The method, according to claim 1, wherein the autoimmune disorder is a neuronal autoimmune disorder.

7. The method, according to claim 1, wherein the autoimmune disorder is selected from autism, multiple sclerosis (MS), autoimmune thyroid disease, or psoriasis.

8. The method, according to claim 1, wherein the subject is a candidate for vaccine composition that comprises an agent that induces autoimmunity.

9. The method, according to claim 1, wherein the vaccine immunizes against a disease selected from the group consisting of pertussis, polio, hepatitis, measles, mumps, rubella, influenza, smallpox, zoster, anthrax, tetanus, rotavirus, rabies, pneumonia, chickenpox, meningococcus, diphtheria, anpapillomavirus, anthrax, plague, encephalitis, pneumococcus, pneumonia, typhus, and typhoid fever.

10. A method for providing customized vaccination, comprising:
a) obtaining a biological sample from a subject who is a candidate for vaccination, wherein the biological sample is obtained before the subject receives the vaccination;
b) measuring a level of soluble amyloid precursor protein-alpha (sAPP-α) protein in the sample;
c) correlating the subject's sAPP-α level to the subject's risk of developing vaccine-induced autoimmune disorder; and
d) administering a customized vaccine to the subject if the subject's sAPP-α level correlates to a higher than normal risk of developing vaccine-induced autoimmune disorder.

11. The method, according to claim 10, wherein step (c) comprises comparing the subject's sAPP-α level to a predetermined reference value, wherein the predetermined reference value is determined based on sAPP-α levels in a population, and wherein an elevated level of sAPP-α in the subject's biological sample, when compared to the predetermined reference value, indicates a high risk of developing vaccine-induced autoimmune disorder.

12. The method, according to claim 10, wherein step (d) comprises at least one of the following:
a) administering a customized vaccine composition comprising a reduced amount of pro-inflammatory adjuvant;
b) administering an anti-inflammatory or immune-suppressive agent;
c) determining the subject's sAPP-α level at multiple time points over time, wherein if, at a time point, the subject's sAPP-α level correlates to a low or normal risk of developing vaccine-induced autoimmune disorder, administering a vaccine composition at said time point; or
d) administering a customized vaccine composition that does not comprise an agent that induces autoimmunity.

13. The method, according to claim 11, wherein the subject is a child or an infant.

14. The method, according to claim 10, wherein the biological sample is a blood sample.

15. The method, according to claim 14, wherein the biological sample is selected from a cord blood, prenatal blood, perinatal blood, or postnatal blood sample.

16. The method, according to claim 10, wherein the autoimmune disorder is a neuronal autoimmune disorder.

17. The method, according to claim 10, wherein the autoimmune disorder is selected from autism, multiple sclerosis (MS), autoimmune thyroid disease, or psoriasis.

18. The method, according to claim 10, wherein the vaccine immunizes against a disease selected from the group consisting of pertussis, polio, hepatitis, measles, mumps, rubella, influenza, smallpox, zoster, anthrax, tetanus, rotavirus, rabies, pneumonia, chickenpox, meningococcus, diphtheria, anpapillomavirus, anthrax, plague, encephalitis, pneumococcus, pneumonia, typhus, and typhoid fever.


SEQUENCE LISTING (2 sequences)

SEQ ID NO: 1 - protein, 670 aa, Homo sapiens
LEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQK

SEQ ID NO: 2 - protein, 1762 aa, Homo sapiens
ACTGATGGTAATGCTGGCCTGCTGGCTGAACCCCAGATTGCCATGTTCTGTGGCAGACTGAACAGCACATGAATGTCCAGAATGGGAAGTGGGATTCAGATCCATCAGGGACCAAAACCTGCATTGATACCAAGGAAGGCATCCTGCAGTATTGCCAAGAAGTCTACCCTGAACTGCAGATCACCAATGTGTAGAAGCCAACCAACCAGTGACCATCCAGAACTGGTGCAAGCGGGGCCGCAAGCAGTGCAAGACCCATCCCCACTTTGTGATTCCCTACCGCTGCTTAGTTGGTGAGTTTGTAAGTGATGCCCTTCTGTTCCTGACAAGTGCAAATTCTTACACCAGGAGAGGATGGATGTTTGCGAAACTCATCTTCACTGGCACACCGTCGCCAAAGAGACATGCAGTGAGAAGAGTACCAACTTGCATGACTACGGCATGTGCTGCCCTGCGGAATTGACAAGTTCCGAGGGGTAGAGTTTGTGTGTTGCCCACTGGCTGAAGAAAGTGACAATGTGGATTCTGCTGATGCGGAGGAGGATGACTCGGATGTCTGGTGGGGCGGAGCAACACAGACTATGCAGATGGGAGTGAAGACAAAGTAGTAGAAGTAGCAGAGGAGGAAGAAGTGGCTGAGGTGGAAGAAGAAGAAGCCGATGATGACGAGGACGATGAGGATGGTGATGAGGTAGAGGAGAGGCTGAGGAACCCTACGAAGAAGCCACAGAGAGAACCACCAGCATTGCCACCACCACCACCACCACCACAGAGTCTGTGGAAGAGGTGGTTCGAGTTCCTACAACAGCAGCCAGTACCCCTGATGCGTTGACAAGTATCTCGAGACACCTGGGGATGAGAATGAACATGCCCATTTCCAGAAAGCCAAAGAGAGGCTTGAGGCCAAGCACCGAGAGAGAATGTCCCAGGTCATGAGAGAATGGGAAGAGGCAAACGTCAAGCAAAGAACTTGCCTAAAGCTGATAAGAAGGCAGTTATCCAGCATTTCCAGGAGAAAGTGGAATCTTTGGAACAGGAAGCAGCCAACGAGAGACAGCAGCTGGTGGAGACACACATGGCAGAGTGGAAGCCATGCTCAATGACCGCCGCCGCCTGGCCCTGGAGAACTACATCACCGCTCTGCAGGCTGTTCCTCCTCGGCCTCGTCACGTGTTCAATATGCTAAAGAAGTATGTCCGCGCAGAACGAAGGACAGACAGCACACCCTAAAGCATTTCGAGCATGTGCGCATGGTGGATCCCAAGAAAGCCGCTCAGATCCGGTCCCAGGTTATGACACACCTCCGTGTGATTTATGAGCGCATGAATCAGTCTTCTCCCTGCTCTACAACGTGCCTGCAGTGGCCGAGGAGATTCAGGATGAAGTTGATGAGCTGCTTCAGAAAGAGCAAAACTATTCAGATGACGTCTTGGCCAACATGATTAGTGAACCAAGGATCAGTACGGAAACGATGCTCTCATGCCATCTTTGACCGAAACGAAAACCACCGTGGAGCTCCTTCCCGTGAATGGAGAGTTCAGCCTGGACGATCTCCAGCCGTGGCATTCTTTTGGGGCTGACTCTGTGCAGCCAACACAGAAAACGAAGTTGAGCCTGTTGATGCCCGCCCTGCTGCCGACCGAGGACTGACCACTCGACCAGGTTCTGGGTTGACAAATATCAAGACGGAGGAGATCTCTGAAGTGAAGATGGATCAGAATTTCGTCATGACTCAGGATATGAAGTTCATCATCAAAAATAG